(19) United States
(12) Patent Application Publication
(10) Pub. No.: US 2025/0188496 A1
(43) Pub. Date: Jun. 12, 2025

(54) PROCESS FOR PRODUCING FERMENTATION PRODUCTS AND BIOGAS FROM STARCH-CONTAINING MATERIALS

(71) Applicant: Novozymes A/S, Bagsvaerd (DK)

(72) Inventors: Lindsey TUCKER, Fort Collins, CO (US); Billy WHITLOCK, Lakewood, CO (US)

(73) Assignee: Novozymes A/S, Bagsvaerd (DK)

(21) Appl. No.: 18/840,367

(22) PCT Filed: Feb. 21, 2023
(86) PCT No.: PCT/US2023/062956

§ 371 Date: Aug. 21, 2024

Related U.S. Application Data
(60) Provisional application No. 63/313,129, filed on Feb. 23, 2022.

(51) Int. Cl. C12P 5/02; C11B 3/00; C12C 11/11; C12F 3/06; C12P 7/14; C12P 21/00

(52) U.S. Cl. CPC C12P 5/023; C11B 3/003; C12C 11/11; C12F 3/06; C12P 7/14; C12P 21/00

(57) ABSTRACT

The present invention concerns a process for producing fermentation products, such as ethanol, and a biogas unit, wherein a yield enhancing composition comprising at least one, at least two, at least three, at least four, or at least five different types of enzymes and/or at least one, at least two, at least three, at least four, or at least five different microorganisms is added to the whole stillage that is fed to the slurrying step and/or the biogas unit, outflow of the biogas unit before being mashed in the slurrying step; thin stillage that is fed to the slurrying step, residual materials resulting from purification of the oil and/or of the protein product that are fed to the biogas unit, wet cake that is fed to the slurrying step and/or fed to the biogas unit, added to the biogas unit, and/or biomass added to any one of the preceding steps.

### BACKGROUND OF THE INVENTION

### Field of the Invention

The invention relates to a process for producing fermentation products and biogas from starch-containing material, wherein a yield enhancing composition comprising at at least one, least two, at least three, at least four, or at least five different types of enzymes and/or at least one, at least two, at least three, at least four, or at least five different microorganism strains is i) added to the whole stillage that is fed to the slurrying step; and/or ii) added to the whole stillage that is fed to the biogas unit; and/or iii) added to the outflow of the biogas unit before being mashed with the milled starch-containing material (e.g., corn from dry milling) in the slurrying step; and/or iv) added to the thin stillage that is fed to the slurrying step; and/or v) added to the residual materials resulting from purification of the oil (e.g., corn) that are fed to the biogas unit; and/or vi) added to the residual materials resulting from purification of the protein product that are fed to the biogas unit; and/or vii) added to the wet cake that is fed to the slurrying step; and/or viii) added to the wet cake that is fed to the biogas unit; and/or ix) added to the biogas unit; and/or x) biomass added to any one of i)-ix).

### Description of the Related Art

The production of ethanol from corn is known and is a technology which is widely used on an industrial scale, particularly in the USA. EP 2 501 818 B1 describes a process in which the stillage from an ethanol unit is fed to a biogas unit and a portion of the outflow from the biogas unit is recycled directly to the ethanol unit. U.S. Pat. No. 8,962,309 B2 describes a biogas unit with different types of biogas fermenters for thin stillage and wet cake. The biogas fermenters therein should be operated at an ammonium concentration of less than 6000 ppm NH4-N. WO 2013/000925 A1 describes a process in which thin stillage is fed to a biogas fermenter of the continuous stirred tank reactor (CSTR) type with a mean hydraulic residence time of 1-20 days. US2022/0033860 describes a method for carrying out the combined operation of a cold cook ethanol production unit and a biogas unit, wherein cellulases or cellulase-forming microorganisms are added to the outflow of a biogas unit that is then recycled to the mashing step.

### SUMMARY OF THE INVENTION

The present invention relates to processes of producing fermentation products, such as ethanol, and biogas from starch-containing material, using a yield enhancing composition comprising at least one, at least two, at least three, at least four, or at least five different types of enzymes and/or at least one, at least two, at least three, or at least four different microbial strains.

The problem to be solved by the invention is to operate a combination of a fermentation product production process (e.g., bioethanol unit) and a biogas unit which saves energy, water and chemicals compared with a typical fermentation product production process (e.g., bioethanol unit) and which, despite the energy savings, has high ethanol yields per tonne of milled starch-containing material (e.g., corn from dry milling) and enhanced ethanol yields and/or biogas yields. In addition, the use of outflow from the biogas unit as process liquids for the fermentation product production process (e.g., bioethanol unit) should not have a negative effect on the ethanol fermentation.

In one aspect, the present invention provides a method for carrying out the combined operation of a process for producing a fermentation product and a biogas, comprising:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and to the biogas unit,
wherein a yield enhancing composition comprising at least one, at least two, at least three, at least four, or at least five different types of enzymes and/or at least one, at least two, at least three, or at least four different microbial strains is:
   i) added to the whole stillage from e) that is fed to the slurrying step in a); and/or
   ii) added to the whole stillage from e) that is fed to the biogas unit; and/or
   iii) added to the outflow of the biogas unit before being fed to slurrying step a); and/or
   iv) added to the thin stillage that is fed to the mashing step; and/or
   v) added to the residual materials resulting from purification of the corn oil that are fed to the biogas unit; and/or
   vi) added to the residual materials resulting from purification of the protein product that are fed to the biogas unit; and/or
   vii) added to the wet cake that is fed to the mashing step; and/or
   viii) added to the wet cake that is fed to the biogas unit; and/or
   ix) added to the biogas unit; and/or
   x) biomass added to any one of i)-ix).

In step a), milled starch-containing material, preferably corn from dry milling, is mashed with liquids to form a slurry. These liquids consist of at least 0.1 t TS of whole stillage and at least 0.1 m³ of outflow from the biogas unit per tonne of milled starch-containing material (e.g., corn from dry milling).

The recycle of outflow from the biogas unit with the yield enhancing composition has the advantage that ethanol yields can be enhanced by providing enzymes and/or microorganisms that can degrade materials in the outflow remaining post anerobic digestion, such as residual starch, cellulose, hemicellulose, yeast cell wall beta-glucan, protein, etc. to produce additional fermentable sugars and nitrogen to fuel the yeast during fermentation, while savings can also be made as regards fresh water as well as process liquids. In a typical ethanol unit, process liquids are obtained in an energy-intensive manner by the evaporation of thin stillage.

In step b), the slurry from step a) is fed to a liquefying step in which the mash is heated to temperatures above the gelatinization temperature of the starch in the starch-containing material.

In step c), the mash from the liquefaction step is fed to a saccharification step in which the dextrins is saccharified to produce fermentable sugars, that are then fed to a fermenting step d) in which a beer comprising a fermentation product (e.g., ethanol) is produced. Steps c) and d) may be performed sequentially or simultaneously.

In step e), the beer containing the fermentation product (e.g., ethanol-containing mash) from the fermentation step is then fed to a distillation step in which the ethanol is separated out to produce whole stillage.

In step f), a portion of the fermentation product depleted mash (e.g., ethanol-depleted mash) from the distillation step, what is known as the whole stillage, is fed without the typical solid-liquid separation directly to the slurrying step a) and to the biogas unit. Compared with typical solid-liquid separation of the whole stillage in its entirety, this means that savings are made as regards the electrical energy for the solid-liquid separation of whole stillage and a large proportion of the residual starch from the fermentation step is recycled which would otherwise no longer be available for fermentation product (e.g., ethanol) formation because it would have been used as animal feed. The addition of a yield enhancing composition comprising at least one, at least two, at least three, at least four, or at least five different types of enzymes and/or at least one, at least two, at least three, at least, or at least five different microbial strains formulated to enhance fermentation product yield (e.g., ethanol) and/or biogas yield is i) added to the whole stillage from b) that is fed to the slurrying step in a); and/or ii) added to the whole stillage from b) that is fed to the biogas unit; and/or iii) added to the outflow of the biogas unit before being mashed with the milled starch-containing material in slurrying step a); and/or iv) added to the thin stillage that is fed to the mashing step; and/or v) added to the residual materials resulting from purification of the corn oil that are fed to the biogas unit; and/or vi) added to the residual materials resulting from purification of the protein product that are fed to the biogas unit; and/or vii) added to the wet cake that is fed to the mashing step; and/or viii) added to the wet cake that is fed to the biogas unit; and/or ix) added to the biogas unit; and/or x) biomass added to any one of i)-ix).

The addition of the yield enhancing composition in this manner further increases fermentation product yield (e.g., ethanol yield), biogas yield, and/or fermentation product yield (e.g., ethanol yield) and biogas yield, and/or the rate of biogas production.

### BRIEF DESCRIPTION OF THE DRAWINGS

The invention will now be described in more detail with the aid of two exemplary embodiments and associated drawings, in which:

FIG. 1 diagrammatically describes a process workflow in accordance with embodiments of the invention.

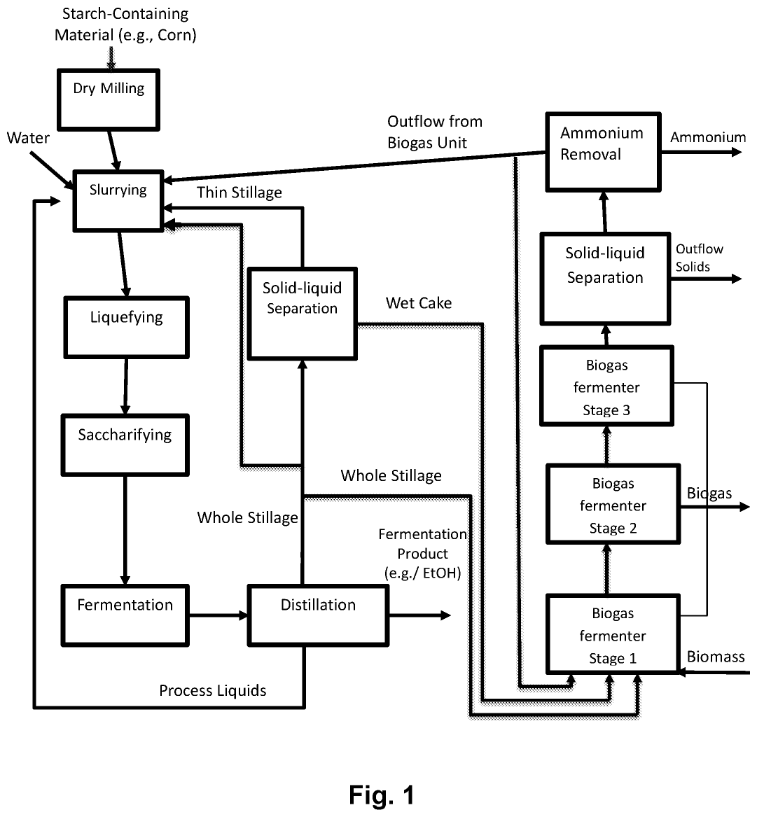

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

As used herein, “initial gelatinization temperature” refers to the lowest temperature at which solubilization of starch, typically by heating, begins. The temperature can vary for different starches.

The term “dry matter” (TS) means the solid residue which is obtained after removing the solvent (for example water or ethanol) from a suspension (for example from stillage) or from a solution. The solid residue includes all of the previously dissolved or suspended solids (for example raw proteins, yeast and salts). The mass of the dry matter is known as the dry mass and can be given in kilograms. The term “dry matter content” (TS content) means the percentage mass fraction of the dry matter with respect to the total mass of the suspension (for example the stillage) or solution.

The term “stillage” means the residue from distillation of an ethanol-containing grain mash. The term “whole stillage” is synonymous with “stillage”.

The term “solid-liquid separation” means a process which separates a suspension (for example stillage) into a two-phase system comprising a solid phase and a liquid phase. The solid-liquid separation step may advantageously be carried out in a separator or decanter. The term “solid phase” should be understood, in a two-phase system, to mean the phase which has the higher dry matter content. A solid phase in this regard may include a suspension or a sedimented solid (residue). The term “liquid phase” in a two-phase system means the phase which has the lower dry matter content. In this regard, a liquid phase may include a suspension or a clear solution.

The term “thin stillage” means a liquid phase produced by solid-liquid separation of whole stillage. The TS content of a thin stillage can advantageously be at least 8 percent. The term “wet cake” means the solid phase which is separated from the stillage by solid-liquid separation.

The term “outflow solid” means the solid phase which is separated from the outflow from a biogas fermenter by solid-liquid separation.

The term “ammonium nitrogen content” (NH4-N content) means the mass fraction of nitrogen in the form of ammonium in a sample. In order to measure the NH4-N content of a sample, the sample is made basic with sodium hydroxide and then distilled with steam. Ammonia is driven out of the sample and trapped in a boric acid receiving tank. The mass fraction of ammonium nitrogen is determined by titration of the borate, which is formed, using dilute hydrochloric acid.

The terms “biomass” as used herein includes any polymeric sugar containing material. Preferably, biomass as used herein includes cellulosic material and/or lignocellulosic material and/or hemicellulosic material and/or starch material and/or pectin material and/or lipid material. Biomass suitable for use in the processes as described herein includes virgin biomass and/or non-virgin biomass such as agricultural biomass, processed agricultural biomass, commercial organics, construction and demolition debris, municipal solid waste, household waste, waste paper and yard waste, waste of the dairy industry (comprising for example lactose), waste from the beet and cane sugar industry (comprising for example sucrose (saccharose)) and by-products of oil seeds (for example rape seed meal, sunflower meal and soy husks). Common forms of biomass include trees, shrubs and grasses, wheat, rye, oat, wheat straw, sugar cane, cane straw, sugar cane bagasse, switch grass, miscanthus, energy cane, cassava, molasse, barley, corn, corn stover, corn fiber, corn husks, corn cobs, canola stems, soybean stems, sweet sorghum, corn kernel including fiber from kernels, distillers dried grains (DDGS), brewery spent grains, silage such as corn silage, grass silage and beet silage, products and by-products from milling of grains such as corn, wheat and barley (including wet milling and dry milling) often called “bran or fibre” as well as municipal solid waste, waste paper and yard waste. The biomass can also be, but is not limited to, herbaceous material, agricultural residues, forestry residues, municipal solid wastes, waste woods (type A, B and/or C), waste paper, and pulp and paper mill residues. “Agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, fruit peels, vines, sugar beet, sugar beet pulp, wheat midlings, oat hulls, and hard and soft woods (not including woods with deleterious materials). In addition, agricultural biomass includes organic waste materials generated from agricultural processes including farming and forestry activities, specifically including forestry wood waste. Agricultural biomass may be any of the afore-mentioned singularly or in any combination or mixture thereof. In a preferred embodiment the biomass is silage.

A “different type of enzyme” means that the enzyme catalyzes a different reaction even though the enzyme may be within the same class. For instance, endoglucanase and cellobiohydrolase catalyze different reactions though they are both cellulases, accordingly endoguclanase and cellobiohydrolase are considered a different type of enzyme according to the instant disclosure.

A “different microorganism strain” means that the strain is from a different genus or species of microorganism, though the microorganism may be the same type of microorganism. For instance, Bacillus subtillis and Bacillus amyloliquefaciens strains are different microorganism even though they are both bacterial strains.

A “yield enhancing composition” is a composition that increases yield by at least 1% within a margin of error of 0.2%. The yield enhancing composition may enhance yield of any product, intermediate product, or co-product described herein. The yield enhancing composition may enhance ethanol yield, biogas yield, protein yield, oil yield, etc.

An a-L-arabinofuranosidase (EC 3.2.1.55) is any polypeptide which is capable of acting on a-L-arabinofuranosides, a-L-arabinans containing (1,2) and/or (1,3)- and/or (1,5)-linkages, arabinoxylans and arabinogalactans. This enzyme may also be referred to as a-N-arabinofuranosidase, arabinofuranosidase or arabinosidase.

An acetyl xylan esterase (EC 3.1.1.72) is any polypeptide which is capable of catalysing the deacetylation of xylans and xylo-oligosaccharides. Such a polypeptide may catalyze the hydrolysis of acetyl groups from polymeric xylan, acetylated xylose, acetylated glucose, alpha-napthyl acetate or p-nitrophenyl acetate but, typically, not from triacetylglycerol. Such a polypeptide typically does not act on acetylated mannan or pectin.

“Amylase” means enzymes that hydrolyze alpha-1,4-glucosidic linkages in starch, both in amylose and amylopectin, such as alpha-amylase (EC 3.2.1.1), beta-amylase (EC 3.2.1.2), glucan 1,4-alpha-glucosidase (EC 3.2.1.3), glucan 1,4-alpha-maltotetraohydrolase (EC 3.2.1.60), glucan 1,4-alpha-maltohexaosidase (EC 3.2.1.98), glucan 1,4-alpha-maltotriohydrolase (EC 3.2.1.116) and glucan 1,4-alpha-maltohydrolase (EC 3.2.1.133), and enzymes that hydrolyze alpha-1,6-glucosidic linkages, being the branch-points in amylopectin, such as pullulanase (EC 3.2.1.41) and limit dextinase (EC 3.2.1.142).

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. As described herein, an expansin-like protein or swollenin-like protein may comprise one or both of such domains and/or may disrupt the structure of cell walls (such as disrupting cellulose structure), optionally without producing detectable amounts of reducing sugars. A cellulose induced protein, for example the polypeptide product of the cip1 or cip2 gene or similar genes (see Foreman et al., J. Biol. Chem. 278(34), 31988-31997, 2003), a cellulose/cellulosome integrating protein, for example the polypeptide product of the cipA or cipC gene, or a scaffoldin or a scaffoldin-like protein. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module (CBM) that mediates attachment of the cellulosome to its substrate. A scaffoldin or cellulose integrating protein may comprise one or both of such domains.

“Glucuronidase” includes enzymes that catalyze the hydrolysis of a glucuronoside, for example b-glucuronoside to yield an alcohol. Many glucuronidases have been characterized and may be suitable for use, for example b-glucuronidase (EC 3.2.1.31), hyalurono-glucuronidase (EC 3.2.1.36), glucuronosyl-disulfoglucosamine glucuronidase (3.2.1.56), glycyrrhizinate b-glucuronidase (3.2.1.128) or a-D-glucuronidase (EC 3.2.1.139).

An “a-D-glucuronidase” (EC 3.2.1.139) is any polypeptide which is capable of catalyzing a reaction of the following form: alpha-D-glucuronoside+H(2)0=an alcohol+D-glucuronate. This enzyme may also be referred to as alpha-glucuronidase or alpha-glucosiduronase. These enzymes may also hydrolyze 4-O-methylated glucuronic acid, which can also be present as a substituent in xylans. An alternative is EC 3.2.1.131: xylan alpha-1,2-glucuronosidase, which catalyses the hydrolysis of alpha-1,2-(4-O-methyl)glucuronosyl links.

A beta-(1,3)(1,4)-glucanase (EC 3.2.1.73) is any polypeptide which is capable of catalyzing the hydrolysis of 1,4-p-D-glucosidic linkages in b-D-glucans containing 1,3- and 1,4-bonds. Such a polypeptide may act on lichenin and cereal b-D-glucans, but not on b-D-glucans containing only 1,3- or 1,4-bonds. This enzyme may also be referred to as licheninase, 1,3-1,4-b-D-glucan 4-glucanohydrolase, b-glucanase, endo-b-1,3-1,4 glucanase, lichenase or mixed linkage b-glucanase. An alternative for this type of enzyme is EC 3.2.1.6, which is described as endo-1,3(4)-beta-glucanase. This type of enzyme hydrolyses 1,3- or 1,4-linkages in beta-D-glucanse when the glucose residue whose reducing group is involved in the linkage to be hydrolysed is itself substituted at C-3. Alternative names include endo-1,3-beta-glucanase, laminarinase, 1,3-(1,3; 1,4)-beta-D-glucan 3 (4) glucanohydrolase. Substrates include laminarin, lichenin and cereal beta-D-glucans.

A coumaroyl esterase (EC 3.1.1.73) is any polypeptide which is capable of catalyzing a reaction of the form: coumaroyl-saccharide+H(2)0=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. This enzyme also falls within EC 3.1.1.73 so may also be referred to as a feruloyl esterase.

“Catalase” means a hydrogen-peroxide: hydrogen-peroxide oxidoreductase (EC 1.11.1.6 or EC 1.11.1.21) that catalyzes the conversion of two hydrogen peroxides to oxygen and two waters. Catalase activity can be determined by monitoring the degradation of hydrogen peroxide at 240 nm based on the following reaction: 2H2O2 (R) 2H2O+O2. The reaction is conducted in 50 mM phosphate pH 7.0 at 25 degrees centigrade with 10.3 mM substrate (H2O2) and approximately 100 units of enzyme per ml. Absorbance is monitored spectrophotometrically within 16-24 seconds, which should correspond to an absorbance reduction from 0.45 to 0.4. One catalase activity unit can be expressed as one micromole of H2O2 degraded per minute at pH 7.0 and 25 degrees centigrade.

A “cellulase” is any polypeptide which is capable of degrading or modifying cellulose. A polypeptide which is capable of degrading cellulose is one which is capable of catalyzing the process of breaking down cellulose into smaller units, either partially, for example into cellodextrins, or completely into glucose monomers. A cellulase as described herein may give rise to a mixed population of cellodextrins and glucose monomers. Such degradation will typically take place by way of a hydrolysis reaction.

“Endoglucanases” are enzymes which are capable of catalyzing the endohydrolysis of 1,4-p-D-glucosidic linkages in cellulose, lichenin or cereal b-D-glucans. They belong to EC 3.2.1.4 and may also be capable of hydrolyzing 1,4-linkages in b-D-glucans also containing 1,3-linkages. Endoglucanases may also be referred to as cellulases, avicelases, b-1,4-endoglucan hydrolases, b-1,4-glucanases, carboxymethyl cellulases, celludextrinases, endo-1,4-b-D-glucanases, endo-1,4-D-glucanohydrolases or endo-1,4-glucanases.

A “beta-glucosidase” (EC 3.2.1.21) is any polypeptide which is capable of catalyzing the hydrolysis of terminal, non-reducing b-D-glucose residues with release of b-D-glucose. Such a polypeptide may have a wide specificity for b-D-glucosides and may also hydrolyze one or more of the following: a b-D-galactoside, an a-L-arabinoside, a b-D-xyloside or a b-D-fucoside. This enzyme may also be referred to as amygdalase, b-D-glucoside glucohydrolase, cellobiase or gentobiase.

A “cellobiohydrolase” (EC 3.2.1.91) is any polypeptide which is capable of catalyzing the hydrolysis of 1,4-D-glucosidic linkages in cellulose or cellotetraose, releasing cellobiose from the ends of the chains. This enzyme may also be referred to as cellulase 1,4-b-cellobiosidase, 1,4-cellobiohydrolase, 1,4-D-glucan cellobiohydrolase, avicelase, bco-1,4-b-0-glucanase, exocellobiohydrolase or exoglucanase.

A “hemicellulase” is any polypeptide which is capable of degrading or modifying hemicellulose. That is to say, a hemicellulase may be capable of degrading or modifying one or more of xylan, glucuronoxylan, arabinoxylan, glucomannan and xyloglucan. A polypeptide which is capable of degrading a hemicellulose is one which is capable of catalyzing the process of breaking down the hemicellulose into smaller polysaccharides, either partially, for example into oligosaccharides, or completely into sugar monomers, for example hexose or pentose sugar monomers. A hemicellulase as described herein may give rise to a mixed population of oligosaccharides and sugar monomers. Such degradation will typically take place by way of a hydrolysis reaction.

An “a-L-arabinofuranosidase” (EC 3.2.1.55) is any polypeptide which is capable of acting on a-L-arabinofuranosides, a-L-arabinans containing (1,2) and/or (1,3)- and/or (1,5)-linkages, arabinoxylans and arabinogalactans. This enzyme may also be referred to as a-N-arabinofuranosidase, arabinofuranosidase or arabinosidase.

An “endo-arabinanase” (EC 3.2.1.99) is any polypeptide which is capable of catalysing endohydrolysis of 1,5-a-arabinofuranosidic linkages in 1,5-arabinans. The enzyme may also be known as endo-arabinase, arabinan endo-1,5-a-L-arabinosidase, endo-1,5-a-L-arabinanase, endo-a-1,5-arabanase; endo-arabanase or 1,5-a-L-arabinan 1,5-a-L-arabinanohydrolase.

An “endoxylanase” (EC 3.2.1.8) is any polypeptide which is capable of catalysing the endohydrolysis of 1,4-p-D-xylosidic linkages in xylans. This enzyme may also be referred to as endo-1,4-p-xylanase or 1,4-p-D-xylan xylanohydrolase. An alternative is EC 3.2.1.136, a glucuronoarabinoxylan endoxylanase, an enzyme that is able to hydrolyze 1,4 xylosidic linkages in glucuronoarabinoxylans.

A “feruloyl esterase (EC 3.1.1.73)” is any polypeptide which is capable of catalyzing a reaction of the form: feruloyl-saccharide+H₂O=ferulate+saccharide. The saccharide may be, for example, an oligosaccharide or a polysaccharide. It may typically catalyze the hydrolysis of the 4-hydroxy-3-methoxycinnamoyl (feruloyl) group from an esterified sugar, which is usually arabinose in ‘natural’ substrates p-nitrophenol acetate and methyl ferulate are typically poorer substrates. This enzyme may also be referred to as cinnamoyl ester hydrolase, ferulic acid esterase or hydroxycinnamoyl esterase. It may also be referred to as a hemicellulase accessory enzyme, since it may help xylanases and pectinases to break down plant cell wall hemicellulose and pectin.

A “pectinase” is any polypeptide which is capable of degrading or modifying pectin. A polypeptide which is capable of degrading pectin is one which is capable of catalyzing the process of breaking down pectin into smaller units, either partially, for example into oligosaccharides, or completely into sugar monomers. A pectinase as described herein may give rise to a mixed population of oligosacchardies and sugar monomers. Such degradation will typically take place by way of a hydrolysis reaction.

An “a-galactosidase” (EC 3.2.1.22) is any polypeptide which is capable of catalyzing the hydrolysis of terminal, non-reducing a-D-galactose residues in a-D-galactosides, including galactose oligosaccharides, galactomannans, galactans and arabinogalactans. Such a polypeptide may also be capable of hydrolyzing a-D-fucosides. This enzyme may also be referred to as melibiase.

A “beta-galactosidase” (EC 3.2.1.23) is any polypeptide which is capable of catalyzing the hydrolysis of terminal non-reducing b-D-galactose residues in b-D-galactosides. Such a polypeptide may also be capable of hydrolyzing a-L-arabinosides. This enzyme may also be referred to as exo-(1->4)-D-galactanase or lactase.

A “beta-mannanase” (EC 3.2.1.78) is any polypeptide which is capable of catalyzing the random hydrolysis of 1,4-D-mannosidic linkages in mannans, galactomannans and glucomannans. This enzyme may also be referred to as mannan endo-1,4-mannosidase or endo-1,4-mannanase.

A “beta-mannosidase” (EC 3.2.1.25) is any polypeptide which is capable of catalyzing the hydrolysis of terminal, non-reducing b-D-mannose residues in b-D-mannosides. This enzyme may also be referred to as mannanase or mannase.

An “endo-polygalacturonase” (EC 3.2.1.15) is any polypeptide which is capable of catalyzing the random hydrolysis of 1,4-a-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-a-1,4-galacturonide glycanohydrolase, endogalacturonase; endo-D-galacturonase or poly(1,4-a-D-galacturonide) glycanohydrolase.

A “pectin methyl esterase” (EC 3.1.1.11) is any enzyme which is capable of catalysing the reaction: pectin+n H₂O=n methanol+pectate. The enzyme may also be known as pectinesterase, pectin demethoxylase, pectin methoxylase, pectin methylesterase, pectase, pectinoesterase or pectin pectylhydrolase.

An “endo-galactanase” (EC 3.2.1.89) is any enzyme capable of catalyzing the endohydrolysis of 1,4-D-galactosidic linkages in arabinogalactans. The enzyme may also be known as arabinogalactan endo-1,4-galactosidase, endo-1,4-galactanase, galactanase, arabinogalactanase or arabinogalactan 4-D-galactanohydrolase.

A “pectin acetyl esterase” is defined herein as any enzyme which has an acetyl esterase activity which catalyzes the deacetylation of the acetyl groups at the hydroxyl groups of GalUA residues of pectin.

An “endo-pectin lyase” (EC 4.2.2.10) is any enzyme capable of catalysing the eliminative cleavage of (1 (R)4)-a-D-galacturonan methyl ester to give oligosaccharides with 4-deoxy-6-O-methyl-a-D-galact-4-enuronosyl groups at their non-reducing ends. The enzyme may also be known as pectin lyase, pectin frans-eliminase; endo-pectin lyase, polymethylgalacturonic transeliminase, pectin methyltranseliminase, pectolyase, PL, PNL or PMGL or (1 (R)4)-6-O-methyl-a-D-galacturonan lyase.

A “pectate lyase” (EC 4.2.2.2) is any enzyme capable of catalysing the eliminative cleavage of (1 (R)4)-a-D-galacturonan to give oligosaccharides with 4-deoxy-a-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, a-1,4-D-endopolygalacturonic acid lyase, PGA lyase, PPase-N, endo-a-1,4-polygalacturonic acid lyase, polygalacturonic acid lyase, pectin frans-eliminase, polygalacturonic acid frans-eliminase or (1 (R)4)-a-D-galacturonan lyase.

An “alpha rhamnosidase” (EC 3.2.1.40) is any polypeptide which is capable of catalysing the hydrolysis of terminal non-reducing a-L-rhamnose residues in a-L-rhamnosides or alternatively in rhamnogalacturonan. This enzyme may also be known as a-L-rhamnosidase T, a-L-rhamnosidase N or a-L-rhamnoside rhamnohydrolase.

An “exo-galacturonase” (EC 3.2.1.82) is any polypeptide capable of hydrolysis of pectic acid from the non-reducing end, releasing digalacturonate. The enzyme may also be known as exo-poly-a-galacturonosidase, exopolygalacturonosidase or exopolygalacturanosidase.

An “exo-galacturonase” (EC 3.2.1.67) is any polypeptide capable of catalysing: (1,4-a-D-galacturonide)n+H₂O=(1,4-a-D-galacturonide)”-i+D-galacturonate. The enzyme may also be known as galacturan 1,4-a-galacturonidase, exopolygalacturonase, poly(galacturonate) hydrolase, exo-D-galacturonase, exo-D-galacturonanase, exopoly-D-galacturonase or poly(1,4-a-D-galacturonide) galacturonohydrolase.

An “exopolygalacturonate lyase” (EC 4.2.2.9) is any polypeptide capable of catalysing eliminative cleavage of 4-(4-deoxy-a-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-frans-eliminase, PATE, exo-PATE, exo-PGL or (1 (R)4)-a-D-galacturonan reducing-end-disaccharide-lyase.

A “rhamnogalacturonan hydrolase” is any polypeptide which is capable of hydrolyzing the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

A “rhamnogalacturonan lyase” is any polypeptide which is any polypeptide which is capable of cleaving a-L-Rhap-(1 (R)4)-a-D-GalpA linkages in an endo-fashion in rhamnogalacturonan by beta-elimination.

A “rhamnogalacturonan acetyl esterase” is any polypeptide which catalyzes the deacetylation of the backbone of alternating rhamnose and galacturonic acid residues in rhamnogalacturonan.

A “rhamnogalacturonan galacturonohydrolase” is any polypeptide which is capable of hydrolyzing galacturonic acid from the non-reducing end of strictly alternating rhamnogalacturonan structures in an exo-fashion.

A “xylogalacturonase” is any polypeptide which acts on xylogalacturonan by cleaving the b-xylose substituted galacturonic acid backbone in an enc/o-manner. This enzyme may also be known as xylogalacturonan hydrolase.

“Ligninase” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin. Ligninases include but are not limited to the following group of enzymes: lignin peroxidases (EC 1.11.1.14), manganese peroxidases (EC 1.11.1.13), laccases (EC 1.10.3.2) and feruloyl esterases (EC 3.1.1.73).

“Hexosyltransferase” (2.4.1-) includes enzymes which are capable of catalysing a transferase reaction, but which can also catalyze a hydrolysis reaction, for example of cellulose and/or cellulose degradation products. An example of a hexosyltransferase which may be used is a b-glucanosyltransferase. Such an enzyme may be able to catalyze degradation of (1,3)(1,4)glucan and/or cellulose and/or a cellulose degradation product.

“Phytase” means any type of phosphatase enzyme that catalyzes the hydrolysis of phytic acid (myo-inositol hexakisphosphate) which is an indigestible, organic form of phosphorus that is found in grains and oil seeds, and releases a usable form of inorganic phosphorus.

“Lytic polysaccharide monooxygenases (LPMOs)” are enzymes that have recently been classified by CAZy in family AA9 (Auxiliary Activity Family 9) LPMOs or family AA10 (Auxiliary Activity Family 10) LPMOs. Lytic polysaccharide monooxygenases are able to open a crystalline glucan structure and enhance the action of cellulases on lignocellulose substrates. They are enzymes having cellulolytic enhancing activity. Lytic polysaccharide monooxygenases may also affect cello-oligosaccharides. According to the latest literature, (see Isaksen et al., Journal of Biological Chemistry, vol. 289, no. 5, p. 2632-2642), proteins named GH61 (glycoside hydrolase family 61 or sometimes referred to EGIV) are lytic polysaccharide monooxygenases. GH61 was originally classified as endoglucanase based on measurement of very weak endo-1,4-d-glucanase activity in one family member but have recently been reclassified by CAZy in family AA9. CBM33 (family 33 carbohydrate-binding module) is also a lytic polysaccharide monooxygenase (see Isaksen et al, Journal of Biological Chemistry, vol. 289, no. 5, pp. 2632-2642). CAZy has recently reclassified CBM33 in the AA10 family.

A “lactase” (EC 3.2.1.21) is an enzyme that hydrolyses terminal, nonreducing beta-D-glucosyl residues with release of beta-D-glucose.

A “sucrase” (EC 3.2.1.26 (invertase)) catalyzes the hydrolysis of sucrose into fructose and glucose.

A “lipase” is an enzyme that catalyzes the hydrolysis of fats (lipids). Examples include, but are not limited to triacylglycerol lipases, phospholipases (such as A1, A2, B, C and D), cutinases and galactolipases.

A “protease” is a protein hydrolyzing or modifying proteins. Examples include, but are not limited to, endo-acting proteases (serine proteases, metalloproteases, aspartyl proteases, thiol proteases), exo-acting peptidases that cleave off one amino acid, or dipeptide, tripeptide, etc. from the N-terminal (aminopeptidases) or C-terminal (carboxypeptidases) ends of the polypeptide chain.

“Beta-xylosidases” (EC 3.2.1.37) are polypeptides which are capable of atalysing the hydrolysis of 1,4-p-D-xylans, to remove successive D-xylose residues from the nonreducing termini. Beta-xylosidases may also hydrolyze xylobiose. Beta-xylosidase may also be referred to as xylan 1,4-p-xylosidase, 1,4-p-D-xylan xylohydrolase, exo-1,4-p-xylosidase or xylobiase.

As used herein and in the appended claims, the singular forms “a,” “or,” and “the” include plural referents unless the context clearly dictates otherwise.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

### DETAILED DESCRIPTION OF THE INVENTION

Aspects of the present invention relate to processes of producing fermentation products, such as ethanol, and biogas, from starch-containing material, using a fermenting organism.

Processes for Producing Fermentation Products and Biogas from Gelatinized Starch-Containing Material

The invention relates to processes for producing fermentation products, especially ethanol, and biogas from starch-containing material, which process includes a liquefaction step and sequentially or simultaneously performed saccharification and fermentation steps.

Consequently, the invention relates to processes for producing fermentation products and biogas from a starch-containing material comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) simultaneously saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and to the biogas unit,
   wherein a yield enhancing composition comprising at least one, at least two, at least three, at least four, or at least five different types of enzymes and/or at least one, at least two, at least three, or at least four different microbial strains is:
      i) added to the whole stillage from e) that is fed to the slurrying step in a); and/or
      ii) added to the whole stillage from e) that is fed to the biogas unit; and/or
      iii) added to the outflow of the biogas unit before being fed to slurrying step a); and/or
      iv) added to the thin stillage that is fed to the mashing step; and/or
      v) added to the residual materials resulting from purification of the corn oil that are fed to the biogas unit; and/or
      vi) added to the residual materials resulting from purification of the protein product that are fed to the biogas unit; and/or
      vii) added to the wet cake that is fed to the mashing step; and/or
      viii) added to the wet cake that is fed to the biogas unit; and/or
      ix) added to the biogas unit; and/or
      x) biomass added to any one of i)-ix).

### Slurrying Step a)

The starch-containing starting material, such as whole grains, preferably corn, may be reduced in particle size, e.g., by milling, in order to open up the structure, to increase surface area, and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry and wet milling are well known in the art of starch processing. According to the present invention dry milling is preferred. In an embodiment, the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment, at least 50%, preferably at least 70%, more preferably at least 80%, especially at least 90% of the starch-containing material fit through a sieve with #6 screen. The aqueous slurry may contain from 10-55 w/w-% dry solids (DS), preferably 25-45 w/w-% dry solids (DS), more preferably 30-40 w/w-% dry solids (DS) of starch-containing material.

### Liquefying Step b)

Liquefying step b) is typically performed with alpha-amylase to convert the starch in the milled material to dextrins, but may be performed with other enzymes that operate optimally at the elevated temperatures typically used during liquefaction, including for example, proteases, glucoamylases, xylanases, endoglucanases, phytases, lipases (e.g., phospholipase or triacylglycertol lipase). Typically, the enzymes used in liquefaction are stable at the temperatures used. The enzymes may initially be added to the aqueous slurry to initiate liquefaction (thinning). In an embodiment only a portion of the enzymes is added to the aqueous slurry, while the rest of the enzymes are added during the liquefaction step.

The temperature during liquefaction step b) may be in the range from 70-100° C., such as between 75-100° C., preferably between 80-100° C., such as between 85-95° C., such as around between 88 and 92° C. In an embodiment, the temperature during liquefaction step i) is at least 80° C. In an embodiment, the temperature during liquefaction step i) is at least 81° C. In an embodiment, the temperature during liquefaction step i) is at least 82° C. In an embodiment, the temperature during liquefaction step i) is at least 83° C. In an embodiment, the temperature during liquefaction step i) is at least 84° C. In an embodiment, the temperature during liquefaction step i) is at least 85° C. In an embodiment, the temperature during liquefaction step i) is at least 86° C. In an embodiment, the temperature during liquefaction step i) is at least 87° C. In an embodiment, the temperature during liquefaction step i) is at least 88° C. In an embodiment, the temperature during liquefaction step i) is at least 89° C. In an embodiment, the temperature during liquefaction step i) is at least 90° C. In an embodiment, the temperature during liquefaction step i) is at least 91° C. In an embodiment, the temperature during liquefaction step i) is at least 92° C. In an embodiment, the temperature during liquefaction step i) is at least 93° C. In an embodiment, the temperature during liquefaction step i) is at least 94° C. In an embodiment, the temperature during liquefaction step i) is at least 95° C. In an embodiment, the temperature during liquefaction step i) is at least 96° C. In an embodiment, the temperature during liquefaction step i) is at least 97° C. In an embodiment, the temperature during liquefaction step i) is at least 97° C. In an embodiment, the temperature during liquefaction step i) is at least 98° C. In an embodiment, the temperature during liquefaction step i) is at least 99° C. In an embodiment, the temperature during liquefaction step i) is at least 100° C.

The liquefaction step b) may be carried out for 0.5-5 hours, such as 1-3 hours, such as typically around 2 hours.

The aqueous slurry may in an embodiment be jet-cooked to further gelatinize the slurry before being subjected to liquefaction in step b). The jet-cooking may be carried out at a temperature between 110-145° C., preferably 120-140° C., such as 125-135° C., preferably around 130° C. for about 1-15 minutes, preferably for about 3-10 minutes, especially around about 5 minutes.

The pH during liquefaction step b) may be between 4-7. In an embodiment, the pH during liquefaction is from 4.5-5.0, such as between 4.5-4.8. In another embodiment liquefaction is carried out at a pH above 5.0-6.5, such as above 5.0-6.0, such as above 5.0-5.5, such as between 5.2-6.2, such as around 5.2, such as around 5.4, such as around 5.6, such as around 5.8.

### Saccharification Step c) and Fermentation Step d)

Saccharification step c) and fermenting step d) may be performed sequentially or simultaneously. A glucoamylase, may be present and/or added during saccharification step c) and/or fermentation step d). Other enzymes may also be present and/or added during saccharification step c) and/or fermenting step d), including, but not limited to, beta-amylase, maltogenic amylase, alpha-glucosidase, alpha-amylase, such as a fungal alpha-amylase, trehalase, protease, cellulase, hemicellulase, etc.

When doing sequential saccharification and fermentation, saccharification step c) may be carried out at conditions well-known in the art. For instance, the saccharification step c) may last up to from about 24 to about 72 hours. In an embodiment, pre-saccharification is done. Pre-saccharification is typically done for 40-90 minutes at a temperature between 30-65° C., typically about 60° C. Pre-saccharification is in an embodiment followed by saccharification during fermentation in simultaneous saccharification and fermentation (“SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step c) and the fermentation step d) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

### Distillation Step e)

The fermentation product containing beer (e.g., ethanol-containing mash) from the fermentation step d) is then fed to a distillation step in which the fermentation product (e.g., ethanol) is separated out. This results in significant energy savings compared with typical distillation steps at approximately 108 degrees centigrade. In a preferred embodiment, the fermentation product containing mash (e.g., ethanol-containing mash) is heated to a maximum of 87 degrees centigrade, preferably to a maximum of 79 degrees centigrade, particularly preferably to a maximum of 68 degrees centigrade. In addition to saving energy, the low temperatures of the distillation step also mean that higher quality proteins are present in the whole stillage. This can be used to obtain high value animal feed and foodstuffs from the whole stillage or thin stillage.

Step f) Feeding the Whole Stillage from Step e) to Slurrying Step a) and to the Biogas Unit

The fermentation product depleted mash (e.g., ethanol-depleted mash) from the distillation step, what is known as the whole stillage, may be re-used in several different ways. A portion is recycled directly to the slurrying step a). A further portion is fed directly to the biogas unit. The direct use of whole stillage has the following advantages over the typical solid-liquid separation of the entirety of the whole stillage: on the one hand, electrical energy for the solid-liquid separation is saved. On the other hand, a large proportion of the residual starch from the fermentation is recycled; as animal feed or as a substrate for the biogas unit, it would no longer be available for ethanol formation.

Fluids such as whole stillage, outflow from the biogas unit, and/or thing stillage or process liquids can be recycled in slurrying step a) to be mashed together with the the milled starch-containing material (e.g., corn from dry milling) to form the aqueous slurry. Because the recycle of dry matter in the form of whole stillage into the slurrying step a) is high, the advantages of the energy savings of the mash distillation at low temperatures can be obtained and at the same time, increased ethanol yield due recycling of additional glucose and nitrogen sources can be obtained. In a preferred embodiment, the per tonne of milled starch-containing material (e.g., corn from dry milling) in the distillation step, at least 400 liters of ethanol, preferably at least 425 liters of ethanol, particularly preferably at least 435 liters of ethanol are separated. In this regard, at least 0.1 t TS in the form of whole stillage per tonne of milled starch-containing material (e.g., corn from dry milling) is recycled to the slurrying step a). In a preferred embodiment, at least 0.12 t TS, preferably at least 0.14 t TS, particularly preferably at least 0.16 t TS in the form of whole stillage per tonne of milled starch-containing material (e.g., corn from dry milling) is recycled to the slurrying step a).

In a preferred embodiment, a yield enhancing composition comprising at least one, at least two, at least three, at least four, or at least five different enzymes and/or at least one, at least two, at least three, at least four or at least five different microorganisms can also be added to the whole stillage that is recycled to the slurrying step a). This has the advantage that glucose and nitrogen sources from the enzymatic hydrolysis of materials remaining in the whole stillage, e.g., hemicellulose, cellulose, residual starch, protein, yeast cell wall beta-glucan etc.) is made available to the yeasts in the fermentation step, and therefore the ethanol yield can be increased. With the high recycle of whole stillage in accordance with the invention compared with a typical fermentation product production process, large quantities of hemicellulose, cellulose, residual starch, protein, yeast cell wall beta-glucan etc. can also be recycled to the slurrying step and thus are available for enzymatic hydrolysis and subsequent assimilation by yeast for improved fermentations.

In a preferred embodiment, biomass is added with the yield enhancing composition to the whole stillage that is recycled to the slurrying step a). The biomass may be pretreated before being added with the yield enhancing composition to the whole stillage that is recycled to the slurrying step. Pretreatment methods are known in the art and include, but are not limited to, heat, mechanical, chemical modification, biological modification and any combination thereof. The biomass may also be washed before it is contacted with the yield enhancing composition. The whole stillage and biomass, e.g., pretreated biomass, may be incubated together with the yield enhancing composition before being recycled to the slurrying step to initiate the hydrolysis of the hemicellulose, cellulose, residual starch, protein, yeast cell wall beta-glucan, etc. to make it more readily available for the yeast during fermentation.

In a further preferred embodiment, at least 0.2 t TS, preferably at least 0.3 t TS, particularly preferably at least 0.4 t TS per tonne of milled starch-containing material (e.g., corn from dry milling) is added to slurrying step a) in the form of a mixture of thin stillage and whole stillage. In a preferred embodiment, the mixture of thin stillage and whole stillage contains a volume fraction of at least 10 percent, preferably at least 20 percent, particularly preferably at least 30 percent whole stillage.

In a preferred embodiment, at least 0.1 t TS, preferably at least 0.2 t TS, preferably at least 0.25 t TS thin stillage pert of mash is recycled to slurrying step a). Recycling larger quantities of thin stillage is advantageous compared with using fresh water or process liquids, which are obtained in an energy-intensive manner by the evaporation of thin stillage.

In a preferred embodiment, at least 15 kg, preferably at least 18 kg, particularly preferably at least 20 kg of glycerin per tonne of starch-containing material (e.g., corn meal), is recycled to the slurrying step via the whole stillage and thin stillage. A high recycle of glycerin significantly reduces the fresh formation of glycerin in the ethanol fermentation step. In this manner, less starch or sugar is lost as glycerin and thus the ethanol yield is increased. In a preferred embodiment, the recycle of the mixture of thin stillage and whole stillage is selected to be so high that a mass fraction of less than 2.5 percent, preferably less than 2.3 percent, particularly preferably less than 2.1 percent of the starch and sugar present in the starch-containing material (e.g., corn from dry milling) is transformed into glycerin. In a preferred embodiment, the recycle of the mixture of thin stillage and whole stillage is selected to be so high that less than 19 kg, preferably less than 17 kg, particularly preferably less than 15 kg of glycerin per tonne of starch-containing material (e.g., corn from dry milling) is produced.

The fraction of outflow from the biogas unit is at least 0.1 m³ per tonne of starch-containing material (e.g., corn meal). Recycling outflow from the biogas unit has the advantage of economizing on fresh water as well as process liquids. Process liquids are typically obtained in a hi an energy-intensive manner by the evaporation of thin stillage. The addition of outflow from the biogas unit to the slurrying step a) does not lead to a severe inhibition of the enzymes and yeasts in the ethanol process if the addition of NH4-N in the form of outflow from the biogas unit is limited. In a preferred embodiment, in the slurrying step a), a maximum of 1000 g, preferably a maximum of 800 g, particularly preferably a maximum of 600 g of NH4-N per tonne of starch-containing material (e.g., corn from dry milling) is recycled via the outflow from the biogas unit. On the other hand, a certain quantity of NH4-IN recycle into the slurrying step a) should be aimed for to reduce or dispense with the use of external nitrogen sources such as urea. In a preferred embodiment, in the slurrying step a), at least 100 g, preferably at least 200 g, particularly preferably at least 400 g of ammonium nitrogen per tonne of starch-containing material (e.g., corn from dry milling) is recycled via the outflow from the biogas unit. In a preferred embodiment, the fraction of outflow from the biogas unit per tonne of starch-containing material (e.g., corn meal) is at least 0.2 m³, preferably at least 0.4 m³, particularly preferably at least 0.8 m³.

In a preferred embodiment, a yield enhancing composition comprising at least one, at least two, at least three, at least four, or at least five different enzymes and/or at least one, at least two, at least three, at least four or at least five different microorganisms can also be fed from the biogas unit to the slurrying step a) via the outflow from the biogas unit. This has the advantage that in this manner, glucose from the enzymatic hydrolysis of materials remaining after anaerobic digestion of the whole stillage and/or biomass, e.g., hemicellulose, cellulose, residual starch, protein, yeast cell wall beta-glucan, etc.) is made available to the yeasts in the fermentation step, and therefore the ethanol yield can be increased. With the high recycle of whole stillage in accordance with the invention compared with a typical ethanol unit, large quantities of hemicellulose, cellulose, residual starch, protein, yeast cell wall beta-glucan, etc. can also be recycled to the slurrying step a) and thus are available for enzymatic hydrolysis.

Preferably, cellulose is present in a mass fraction of at least 5 percent of the TS of the whole stillage. In an embodiment, the cellulose is present in a mass fraction of from 5 percent to 20 percent of the TS of the whole stillage. The recycle of at least 0.1 t TS to the slurrying step a) therefore recycles at least 5 kg of cellulose. In an embodiment, the recycle of at least 0.1 t TS to the slurrying step a) recycles from 5 kg to 20 kg of cellulose. The cellulose content in the whole stillage is determined by means of an animal feed analysis in accordance with VDLUFA Ill. The cellulose content is calculated as the measured ADF value (“acid detergent fiber”) minus the measured ADL value (“acid detergent lignin”) of a whole stillage sample.

Preferably, hemicellulose is present in a mass fraction of at least 5 percent of the TS of the whole stillage. In an embodiment, the hemicellulose is present in a mass fraction of from 5 percent to 20 percent of the TS of the whole stillage. The recycle of at least 0.1 t TS to the slurrying step a) therefore recycles at least 5 kg of hemicellulose. In an embodiment, the recycle of at least 0.1 t TS to the slurrying step a) recycles from 5 kg to 20 kg of hemicellulose. Methods of determining the hemicellulose content in the whole stillage are well known to those skilled in the art.

Preferably, residual starch is present in a mass fraction of at least 2 percent of the TS of the whole stillage. In an embodiment, the residual starch is present in a mass fraction of from 2 percent to 10% of the TS of the whole stillage. The recycle of at least 0.1 t TS to the slurrying step a) therefore recycles at least 2 kg of residual starch. In an embodiment, the recycle of at least 0.1 T TS to the slurrying step a) recycles from 2 kg to 10 kg of residual starch. Methods of calculating total residual starch are well known to those skilled in the art. A commercial kit for determining residual starch is available for purchase from MEGAZYME®.

Preferably, protein is present in a mass fraction of at least 6 percent of the TS of the whole stillage. In an embodiment, the protein is present in a mass fraction of from 6 percent to 30 percent of the TS of the whole stillage. The recycle of at least 0.1 t TS to the slurrying step a) therefore recycles at least 6 kg of protein. In an embodiment, the recycle of at least 0.1 t TS to the slurrying step a) recycles from 6 kg to 30 kg of protein. When protease are added to the whole stillage prior to recycle, the proteases can break down the protein into amino acids and short oligopeptides that provide a source of free amino nitrogen that is readily assimilable by yeast to fuel yeast growth and enhance fermentation performance. Methods of determining protein content in the whole stillage are well known to those skilled in the art. The residual protein content may be calculated as (protein=nitrogen×6.25) using a LECO FP628 nitrogen analyzer.

Preferably, beta-glucan from yeast cell walls is present in a mass fraction of at least 1 percent of the TS of the whole stillage. In an embodiment, beta-glucan from yeast cell walls is present in a mass fraction of from 1 percent to 10 percent. The recycle of at least 0.1 t TS to the slurrying step a) therefore recycles at least 1 kg of yeast cell wall beta-glucan. The beta-glucanases disclosed herein can be used to hydrolyze the yeast cell wall beta-glucan present in the whole stillage to glucose. Methods for determining yeast cell wall beta-glucan content in the whole stillage are well known to those skilled in the art.

In a further embodiment, process liquids in addition to whole stillage and outflow from the biogas unit are used in the slurrying step a). The process liquids in this regard may be selected from the group comprising: thin stillage, singlings, untreated water, drinking water, water for industrial use, rainwater, ground water, surface water, condensates from the evaporation of thin stillage, process water from CO₂ scrubbers, blowdown water from cooling towers, blowdown water and blow-off water from steam production boilers, and mixtures thereof. In a preferred embodiment, less than 1.2 m³, preferably less than 0.8 m³, particularly preferably less than 0.2 m³ of fresh water is used per tonne of starch-containing material (e.g., corn from dry milling). In such embodiment, a yield enhancing composition comprising at least one, at least two, at least three, at least four, or at least five different enzymes and/or at least one, at least two, at least three, at least four or at least five different microorganisms may be added to the process liquids, the whole stillage, and/or the outflow from the biogas unit.

Step g) Subjecting Whole Stillage from Step f) to a Solid-Liquid Separation Step to Produce Thin Stillage and Wet Cake

In a preferred embodiment, a further portion of the whole stillage is fed to a solid-liquid separation step to produce thin stillage. In a preferred embodiment, a maximum volume fraction of 70 percent, preferably a maximum of 50 percent, particularly preferably a maximum of 30 percent of the whole stillage is fed to a solid-liquid separation step to generate thin stillage and wet cake. As an example, the thin stillage may be used in the slurrying step a), but also for obtaining oil (e.g., corn oil), animal feed or foodstuffs. In a further preferred embodiment, a further portion of the whole stillage is used as animal feed. The wet cake may also be used as animal feed. In a further preferred embodiment, a maximum of 0.18 t TS, preferably a maximum of 0.12 t TS, particularly preferably a maximum of 0.06 t TS of animal feed per tonne of milled starch-containing material (e.g., corn from dry milling) selected from the group comprising whole stillage, wet cake, syrup (thin stillage concentrate) and their dried forms are produced. In a preferred embodiment, the wet cake is fed to the biogas unit.

In a further preferred embodiment, a protein product with a raw protein content (mass fraction of raw protein with respect to TS protein product) of more than 44 percent is obtained from the thin stillage. In a further preferred embodiment, a protein product with a raw protein content (mass fraction of raw protein with respect to TS protein product) of more than 70 percent is obtained from thin stillage. In a further embodiment, oil (e.g., corn oil) is obtained from thin stillage. In a preferred embodiment, the residual materials from the production of protein products and residual materials from the production of oil (e.g., corn oil) are fed to the biogas unit.

Biogas Unit Coupled to Process for Producing Product from Starch-Containing Material

The biogas unit consists of at least one biogas fermenter, for example, of the continuous stirred tank reactor (CTSR) type, in which components of the substrate which is fed in are transformed by a mixed culture of bacteria and archaea to form biogas and by-products such as ammonium. The substrate which is fed in contains at least whole stillage, and in a preferred embodiment may also contain wet cake as well as residual substances from the production of corn oil and from the production of protein products.

In a preferred embodiment, the yield enhancing composition comprising at least one, at least two, at least three, at least four, or at least five different enzymes and/or at least one, at least two, at least three, at least four or at least five different microorganisms can also be added to the whole stillage that is fed to the biogas unit.

In a preferred embodiment, the yield enhancing composition comprising at least one, at least two, at least three, at least four, or at least five different enzymes and/or at least one, at least two, at least three, at least four or at least five different microorganisms can also be added to the wet cake that is fed to the biogas unit.

In a preferred embodiment, the yield enhancing composition comprising at least one, at least two, at least three, at least four, or at least five different enzymes and/or at least one, at least two, at least three, at least four or at least five different microorganisms can also be added to the residual materials from the production of protein products that is fed to the biogas unit.

In a preferred embodiment, the yield enhancing composition comprising at least one, at least two, at least three, at least four, or at least five different enzymes and/or at least one, at least two, at least three, at least four or at least five different microorganisms can also be added to the residual materials from the production of corn oil that is fed to the biogas unit.

In a preferred embodiment, the yield enhancing composition comprising at least one, at least two, at least three, at least four, or at least five different enzymes and/or at least one, at least two, at least three, at least four or at least five different microorganisms is added to the biogas unit.

Depending on the selected process parameters, specific unwanted metabolites may accumulate during the anaerobic fermentation. These are, inter alia, organic acids such as, for example, acetic acid, propionic acid, butyric acid, isobutyric acid, valeric acid, isovaleric acid and caproic acids, and aromatic components such as, for example, phenol, indole, skatole or cresols. They could have a negative effect on the fermentation product production (e.g., ethanol) in the fermentation product production process (e.g., ethanol) and result in odor-related problems.

High ammonium concentrations can inhibit anaerobic fermentation. Typically, then, the NH4-N content is regulated to less than 6000 μm, for example by dilution with process liquids. However, dilution drops the mean residence time of the whole stillage to be fermented in the biogas fermenter, which could result in an unwanted drop in the biogas yield or an accumulation of metabolites. A stable and efficient anaerobic fermentation of whole stillage is also possible at higher ammonium concentrations. This has the advantage that over-dilution and the associated reduction in the residence time, energy consumption and water consumption are reduced. In a preferred embodiment, the ammonium concentration in the biogas fermenters is adjusted to between 6000-9000 ppm, preferably to between 7000-9000 ppm, particularly preferably to between 7500-9000 ppm. In a further preferred embodiment, the temperature in the biogas fermenters in the biogas unit is adjusted to below 43 degrees centigrade. This is advantageous, because at higher temperatures, the chemical equilibrium is displaced from ammonium to ammonia and ammonia has a more inhibiting action on anaerobic fermentation.

Even at high ammonium concentrations, a stable fermentation as well as low contents of unwanted metabolites can be obtained by a cascade configuration of biogas fermenters. In a preferred embodiment, at least three, preferably at least four biogas fermenters are operated in a cascade in the biogas unit. Infeeding is carried out into the first two stages of the cascade or preferably, into only the first stage of the cascade.

To guarantee a stable fermentation as well as low unwanted metabolite contents even at high ammonium concentrations, a sufficiently long mean hydraulic residence time in the biogas unit is vital. In a preferred embodiment, the mean hydraulic residence time for the biogas unit is at least 30 days, preferably at least 50 days, particularly preferably at least 70 days. In a preferred embodiment, the hydraulic residence time is selected to be as long as is required for the concentration of organic acids and aromatic compounds in the outflow from the biogas unit to be a maximum of 450 ppm respectively, preferably a maximum of 300 ppm respectively, particularly preferably a maximum of 150 ppm respectively.

In a further embodiment, the outflow from the biogas fermenter undergoes solid-liquid separation and the solid phase, the outflow solids, is discharged. In a preferred embodiment, the outflow solids are used for fertilizer and soil improvement.

In a preferred embodiment, the outflow from the biogas fermenter or, preferably, the liquid phase from solid-liquid separation of the outflow from the biogas fermenter, is fed to an ammonia stripping step and the NH4-N content of the liquid phase is reduced to less than 1000 ppm, preferably to less than 750 ppm, particularly preferably to less than 500 ppm. This has the advantage that more outflow from the biogas unit can be recycled to the slurrying step without compromising the fermentation product production (e.g., ethanol).

In a further embodiment, the liquid, ammonium-depleted phase from the ammonia stripping step undergoes an evaporation step. In a preferred embodiment, 0.7 t, preferably 0.8 t, particularly preferably 0.9 t of water per tonne of liquid phase is removed from the liquid, ammonium-depleted phase as an evaporation condensate. This high water extraction is advantageous in that a nutrient-rich concentrate, hereinafter termed the nutrient concentrate, is produced which can be used as a valuable fertilizer. Furthermore, less suspended dry matter is recycled to the fermentation product production process (e.g., ethanol) via the outflow from the biogas unit, which reduces the viscosity of the mash or gives rise to larger quantities of milled starch-containing material (e.g., corn from dry milling) or a higher recycle of whole stillage and/or thin stillage. Evaporation of this type is also energetically advantageous compared with typical thin stillage evaporation for the production of process water, because more efficient heat recovery can be carried out.

In a further preferred embodiment, biomass can be fed to the biogas unit as a substrate in addition to stillage, the wet cake or the residual substances from the fermentation product production process (e.g., ethanol). Compared with mono-fermentation of these substances, this has the advantage that the addition of nutrients such as, for example, sodium or nitrogen which are vital to the growth of microorganisms in the biogas unit, is reduced, because these are already present in the residual substances from the fermentation product production process (e.g., ethanol).

The biomass may be pretreated before it is contacted with the yield enhancing composition. Pretreatment methods are known in the art and include, but are not limited to, heat, mechanical, chemical modification, biological modification and any combination thereof. The biomass may also be washed before it is contacted with the yield enhancing composition.

In a further embodiment, a portion of the biogas from the biogas unit is used for the production of process energy for the fermentation product production process (e.g., ethanol) and/or the biogas unit. As an example, biogas can be converted into electrical process energy in a co-generation unit. As a further example, biogas can be used in a steam boiler for the production of steam. In a preferred embodiment, the entire steam requirement for the combined fermentation product production process (e.g., ethanol) and biogas unit can be provided by biogas produced in the biogas unit. In a preferred embodiment, the entire energy requirement for the combined fermentation product production process (e.g., ethanol) and biogas unit is provided by the biogas produced in the biogas unit, wherein the usual units of the prior art may be used for the production of electrical energy and steam from biogas.

In a preferred embodiment, the combined process for producing a fermentation product and a biogas via the biogas unit may be configured such that a maximum of 0.5 m³, preferably a maximum of 0.2 m³, particularly preferably a maximum of 0.1 m³ of effluent is produced per tonne of milled starch-containing material (e.g., corn from dry milling), which effluent is discharged from the combined fermentation product production process (e.g., bioethanol unit) and biogas unit.

In a preferred embodiment, the biogas is fed to a biogas purification step in which at least CO₂ and biomethane are obtained as products. As an example, the biomethane may be compressed and fed into a natural gas grid or used as a vehicle fuel.

### Pretreatment

In one embodiment the biomass (e.g., cellulosic material and/or lignocellulosic material and/or hemicellulosic material and/or starch material and/or pectin material and/or lipid material) is pretreated.

In practicing the processes described herein, any pretreatment process known in the art can be used to disrupt plant cell wall components of the cellulosic-containing material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The biomass material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In a one embodiment, the biomass material is pretreated before saccharification (i.e., hydrolysis) and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

In one embodiment, the biomass material is pretreated with steam. In steam pretreatment, the biomass material is heated to disrupt the plant cell wall components, including lignin, hemicellulose, and cellulose to make the cellulose and other fractions, e.g., hemicellulose, accessible to enzymes. The biomass material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the biomass material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

In one embodiment, the biomass material is subjected to a chemical pretreatment. The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the biomass (e.g., cellulosic-containing material) is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115). In a specific embodiment the dilute acid pretreatment of biomass (e.g., cellulosic-containing material) is carried out using 4% w/w sulfuric acid at 180° C. for 5 minutes.

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment. Lime pretreatment is performed with calcium oxide or calcium hydroxide at temperatures of 85-150° C. and residence times from one hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO2006/032282).

Ammonia fiber expansion (AFEX) involves treating the biomass (e.g., cellulosic-containing) material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the biomass (e.g., cellulosic-containing) material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one embodiment, the chemical pretreatment is carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the biomass (e.g., cellulosic-containing) material and held at a temperature in the range of preferably 140-200° C., e.g., 165-190° C., for periods ranging from 1 to 60 minutes.

In another embodiment, pretreatment takes place in an aqueous slurry. In preferred aspects, the biomass (e.g., cellulosic-containing) material is present during pretreatment in amounts preferably between 10-80 wt. %, e.g., 20-70 wt. % or 30-60 wt. %, such as around 40 wt. %. The pretreated biomass (e.g., cellulosic-containing) material can be unwashed or washed using any method known in the art, e.g., washed with water.

In one embodiment, the biomass material is subjected to mechanical or physical pretreatment. The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The biomass material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in one embodiment, the biomass material is subjected to physical (mechanical) or chemical pretreatment, or any combination thereof, to promote the separation and/or release of cellulose, hemicellulose, and/or lignin.

In one embodiment, the biomass material is subjected to a biological pretreatment. The term “biological pretreatment” refers to any biological pretreatment that promotes the separation and/or release of cellulose, hemicellulose, and/or lignin from the biomass (e.g., cellulosic-containing) material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, DC, 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, DC, chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Fermentation Medium

“Fermentation media” or “fermentation medium” refers to the environment in which fermentation is carried out. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA. 10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cell or fermenting organism may be Saccharomyces strain, e.g., Saccharomyces cerevisiae strain produced using the method described and concerned in U.S. Pat. No. 8,257,959-BB. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The strain may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference), strain no. NRRL Y67342 (See, WO2018/098381 incorporated herein by reference), strain nos. NRRL Y67549 and NRRL Y67700 (See, PCT/US2019/018249 incorporated herein by reference), or any strain described in WO2017/087330 (incorporated herein by reference).

### Starch-Containing Material

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley. In a preferred embodiment, the starch-containing material in a process for producing a fermentation product, wherein the fermentation product is ethanol, is corn or wheat.

### Fermentation Products

The term “fermentation product” means a product produced by a process including a fermentation step using a fermenting organism. A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one embodiment, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603. In one embodiment, the fermentation product is ethanol.

In another embodiment, the fermentation product is an alkane. The alkane may be an unbranched or a branched alkane. The alkane can be, but is not limited to, pentane, hexane, heptane, octane, nonane, decane, undecane, or dodecane. In another embodiment, the fermentation product is a cycloalkane. The cycloalkane can be, but is not limited to, cyclopentane, cyclohexane, cycloheptane, or cyclooctane. In another aspect, the fermentation product is an alkene. The alkene may be an unbranched or a branched alkene. The alkalkaneene can be, but is not limited to, pentene, hexene, heptene, or octene.

In another aspect, the fermentation product is an amino acid. The amino acid can be, but is not limited to, aspartic acid, glutamic acid, glycine, lysine, serine, or threonine. See, for example, Richard and Margaritis, 2004, Biotechnology and Bioengineering 87(4): 501-515.

In another embodiment, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another embodiment, the fermentation product is antibiotics (e.g., penicillin and tetracycline).

In another embodiment, the fermentation product is isoprene.

In another embodiment, the fermentation product is an enzyme.

In another embodiment, the fermentation product is a hormone.

In another embodiment, the fermentation product is a ketone. The term “ketone” encompasses a substance that contains one or more ketone moieties. The ketone can be, but is not limited to, acetone.

In another embodiment, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another embodiment, the fermentation product is polyketide.

In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented biomass (e.g., cellulosic) material or fermented starch-containing material and purified by conventional methods of distillation. As another example, the desired fermentation product may be extracted from the fermentation medium by micro or membrane filtration techniques. Ethanol with a purity of up to about 96 vol. % can be obtained, which can be used as, for example, fuel ethanol, drinking ethanol, i.e., potable neutral spirits, or industrial ethanol.

In some embodiments of the methods, the fermentation product after being recovered is substantially pure. With respect to the methods herein, “substantially pure” intends a recovered preparation that contains no more than 15% impurity, wherein impurity intends compounds other than the fermentation product (e.g., ethanol). In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose orxylose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

### Yield Enhancing Composition of the Present Invention

As noted above, a yield enhancing composition comprising at least one, at least two, at least three, at least four, or at least five different types of enzymes and/or at least one, at least two, at least three, or at least four different microorganism strains may be:
i) added to the whole stillage that is fed to the mashing step; and/or
ii) added to the whole stillage that is fed to the biogas unit; and/or
iii) added to the outflow of the biogas unit before being mashed with the milled starch-containing material in the slurrying step; and/or
iv) added to the thin stillage that is fed to the slurrying step; and/or
v) added to the residual materials resulting from purification of the corn oil that are fed to the biogas unit; and/or
vi) added to the residual materials resulting from purification of the protein product that are fed to the biogas unit; and/or
vii) added to the wet cake that is fed to the slurrying step; and/or
viii) added to the wet cake that is fed to the biogas unit; and/or
ix) added to the biogas unit; and/or
x) biomass added to any one of i)-ix).

The yield enhancing composition may be formulated to enhance biogas yield, fermentation product yield (e.g., ethanol yield), or fermentation product yield (e.g., ethanol yield) and biogas yield. The skilled artisan will appreciate that the manner in which the yield enhancing composition is configured to enhance biogas yield, fermentation product yield (e.g., ethanol yield), or fermentation product yield (e.g., ethanol yield) and biogas yield depends on a variety of factors, including for example, where the yield enhancing composition is being added (e.g., as outlined in i), ii), iii), iv), v), vi), vii), viii), ix), etc.), the composition of the material to which it is added (e.g., cellulose, hemicellulose, protein, fat, yeast cell wall beta-glucan, residual starch, etc.), the type of milled starch-containing material, whether additional biomass is added, the nature and extent to which the biomass has been pre-treated, etc.

The yield enhancing composition may include at least one, at least two, at least three, at least four, or at least five different types of enzymes. The enzymes may be fungal enzymes, bacterial enzymes, or archaeal enzymes. Examples of different types of enzymes suitable for use in the yield enhancing composition include, but are not limited to, an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, and a catalase. In an embodiment, the yield enhancing composition comprises at least one enzyme selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least two different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least three different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least four different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least five different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least six different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least seven different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least eight different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least nine different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. In an embodiment, the yield enhancing composition comprises at least ten different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase. The preceding yield enhancing compositions comprising cellulases and hemicellulases may further include at least one, at least two, at least three, at least four, or at least five different microorganisms (e.g., microbial strains, bacterial strains, mixtures, etc.).

In a preferred embodiment, the yield enhancing composition comprises a cellulase and a hemicellulase. In a preferred embodiment, the yield enhancing composition comprises a cellulase and a hemicellulase, wherein the cellulase is selected from the group consisting of a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least one hemicellulase, wherein the cellulases are selected from the group consisting of a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least one hemicellulase, wherein the cellulases comprise a cellobiohydrolase, an endoglucanase, and a beta-glucosidase and wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least two hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least two hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least two cellulases and at least three hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least three cellulases and at least three hemicellulases, wherein the cellulases are selected from the group consisting of a cellobiohydrolase, an endoglucanase, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least four cellulases and at least one hemicellulases, wherein the cellulases are selected from the group consisting of a cellobiohydrolase I, a cellobiohydrolase II, an endoglucanase I, an endoglucanase II, and a beta-glucosidase and wherein the hemicellulases are selected from the group consisting of endoxylanase, a beta-xylosidase, a aarabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In an embodiment, the yield enhancing composition comprises at least five cellulases and at least one hemicellulase, wherein the cellulases comprise a cellobiohydrolase I, a cellobiohydrolase II, an endoglucanase I, an endoglucanase II, and a beta-glucosidase and wherein the hemicellulases are selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and/or a beta-mannosidase.

In a preferred embodiment, the yield enhancing composition comprises a cellulase and a xylanase. In an embodiment, the yield enhancing composition comprises a cellobiohydrolase and a xylanase. In an embodiment, the yield enhancing composition comprises a beta-glucosidase and a xylanase. In an embodiment, the yield enhancing composition comprises an endoglucanase and a xylanase. In an embodiment, the yield enhancing composition comprises a cellobiohydrolase, a beta-glucosidase and a xylanase. In an embodiment, the yield enhancing composition comprises a cellobiohydrolase, an endoglucanse and a xylanase. In an embodiment, the yield enhancing composition comprises an endoglucanase, a beta-glucosidase and a xylanase. In an embodiment, the yield enhancing composition comprises a cellobiohydrolase, a beta-glucosidase, an endoglucanase and a xylanase. In a preferred embodiment, the yield enhancing composition comprises a cellobiohydrolase I, a beta-glucosidase, an endoglucanase I, and a xylanase. In an embodiment, the yield enhancing composition comprises a cellobiohydrolase I, a beta-glucosidase, an endoglucanase II, and a xylanase. In an embodiment, the yield enhancing composition comprises a cellobiohydrolase II, a beta-glucosidase, an endoglucanase I, and a xylanase. In an embodiment, the yield enhancing composition comprises a cellobiohydrolase I, a beta-glucosidase, an endoglucanase II, and a xylanase. In a preferred embodiment, the yield enhancing composition comprises a cellobiohydrolase I, cellobiohydrolase II, a beta-glucosidase, an endoglucanase I, and a xylanase. In a preferred embodiment, the yield enhancing composition comprises a cellobiohydrolase I, cellobiohydrolase II, a beta-glucosidase, an endoglucanase I, an endoglucanase II, and a xylanase. In an embodiment, the yield enhancing composition comprises a cellobiohydrolase I, cellobiohydrolase II, a beta-glucosidase, an endoglucanase II, and a xylanase. In a preferred embodiment, the yield enhancing composition comprises a cellobiohydrolase II, a beta-glucosidase, an endoglucanase I, and a xylanase. The preceding yield enhancing compositions comprising cellulases and xylanases may further include at least one, at least two, at least three, at least four, or at least five different microorganisms (e.g., microbial strains, bacterial strains, mixtures, etc.).

In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase,

In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microorganisms. In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microbial strains. In a preferred embodiment, the yield enhancing composition comprises a cellulase, a hemicellulase, and a pectinase. In a preferred embodiment, the yield enhancing composition comprises a cellulase, a hemicellulase, and a pectinase, wherein the cellulase is selected from the group consisting of a cellobiohydrolase, an endoglucanase, and a beta-glucosidase, wherein the hemicellulase is selected from the group consisting of endoxylanase, a beta-xylosidase, a arabinofuranosidase a glucuronidase, an acetyl xylan esterase, a feruloyl esterase, a coumaroyl esterase, an alpha-galactosidase, a beta-galactosidase, a beta-mannanase and a beta-mannosidase, wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different fungal strains.

In a preferred embodiment, the yield enhancing composition comprises a beta-glucanase and a pectinase. In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microorganisms.

In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different microbial strains. In a preferred embodiment, the yield enhancing composition comprises a beta-glucanase and a pectinase, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, and at least one, at least two, at least three, at least four, or at least five different fungal strains.

In a preferred embodiment, the yield enhancing composition comprises a beta-glucanase and a pectinase, and at least one, at least two, at least three, at least four, or at least five different microorganisms. In a preferred embodiment, the yield enhancing composition comprises a beta-glucanase and a pectinase, and at least one, at least two, at least three, at least four, or at least five different microorganisms, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, and at least one, at least two, at least three, at least four, or at least five different microbial strains, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase, In a preferred embodiment, the yield enhancing composition comprises a beta-glucanase and a pectinase, and at least one, at least two, at least three, at least four, or at least five different fungal strains, wherein the beta-glucanase is selected from the group consisting of a beta-(1,3)-glucanase, a beta-(1,4)-glucanase a beta-(1,3)(1,4)-glucanase, and a beta-(1,6)-glucanase, and wherein the pectinase is selected from the group consisting of an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and a xylogalacturonase,

In a preferred embodiment, the yield enhancing composition comprises a cellulase, xylanase, protease and lipase. In a preferred embodiment, the yield enhancing composition comprises a cellulase, xylanase, protease and lipase, and at least one, at least two, at least three, at least four, or at least five different microorganisms. In a preferred embodiment, the yield enhancing composition comprises a cellulase, xylanase, protease and lipase, and at least one, at least two, at least three, at least four, or at least five different microbial strains. In a preferred embodiment, the yield enhancing composition comprises a cellulase, xylanase, protease and lipase, and at least one, at least two, at least three, at least four, or at least five different fungal strains.

In a preferred embodiment, the yield enhancing composition comprises a cellulase, amylase, protease and lipase. In a preferred embodiment, the yield enhancing composition comprises a cellulase, amylase, protease and lipase, and at least one, at least two, at least three, at least four, or at least five different microorganisms. In a preferred embodiment, the yield enhancing composition comprises a cellulase, amylase, protease and lipase, and at least one, at least two, at least three, at least four, or at least five different microbial strains. In a preferred embodiment, the yield enhancing composition comprises a cellulase, amylase, protease and lipase, and at least one, at least two, at least three, at least four, or at least five different fungal strains.

In a preferred embodiment, the yield enhancing composition comprises a cellulase, alpha-amylase, protease and triacylglycerol lipase. In a preferred embodiment, the yield enhancing composition comprises a cellulase, alpha-amylase, protease and triacylglycerol lipase, and at least one, at least two, at least three, at least four, or at least five different microorganisms. In a preferred embodiment, the yield enhancing composition comprises a cellulase, alpha-amylase, protease and triacylglycerol lipase, and at least one, at least two, at least three, at least four, or at least five different microbial strains. In a preferred embodiment, the yield enhancing composition comprises a cellulase, alpha-amylase, protease and triacylglycerol lipase, and at least one, at least two, at least three, at least four, or at least five different fungal strains.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase. In a preferred embodiment, the yield enhancing composition at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different microorganisms. In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different microbial strains. In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one, at least two, at least three, at least four, or at least five different fungal strains.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase. In a preferred embodiment, the yield enhancing composition at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different microorganisms. In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different microbial strains. In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one, at least two, at least three, at least four, or at least five different fungal strains.

In a preferred embodiment, the yield enhancing composition comprises an endoglucanase (EG), a cellobiohydrolase (CBH), an endoxylanase (EX), a beta-xylosidase (BX) and a beta-glucosidase (BG). The xylanase may be a GH5 xylanase, a GH10 xylanase, or a GH11 xylanase. The endoglucanase may be a GH5 endoglucanase, a GH7 endoglucanase, or a mixture of a GH5 endoglucanase and a GH7 endoglucanase. The beta-xylosidase may be a GH3 beta-xylosidase. The cellobiohydrolase may be a CBHI or a CHBlI.

In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different microorganisms. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different microbial strains. In a preferred embodiment, the yield enhancing composition comprises at least one, at least two, at least three, at least four, at least five, at least six, or at least seven different enzyme types selected from the group consisting of an amylase, a beta-glucanases, a cellulase, a hemicellulase, a lipase, a pectinase, and a protease, and at least one, at least two, at least three, at least four, or at least five different fungal strains. The amylase may be an alpha-amylase, a glucoamylase, or a combination of alpha-amylase and glucoamylase.

In a preferred embodiment, the yield enhancing composition comprises an alpha-amylase and a glucoamylase. In a preferred embodiment, the yield enhancing composition comprises an alpha-amylase and a glucoamylase and at least one, at least two, at least three, at least four, or at least five different microorganisms. In a preferred embodiment, the yield enhancing composition comprises an alpha-amylase and a glucoamylase and at least one, at least two, at least three, at least four, or at least five different microbial strains. In a preferred embodiment, the yield enhancing composition comprises an alpha-amylase and a glucoamylase and at least one, at least two, at least three, at least four, or at least five different fungal strains.

In a preferred embodiment, the yield enhancing composition comprises a protease. In a preferred embodiment, the yield enhancing composition comprises a protease and at least one, at least two, at least three, at least four, or at least five different microorganisms. In a preferred embodiment, the yield enhancing composition comprises a protease and at least one, at least two, at least three, at least four, or at least five different microbial strains. In a preferred embodiment, the yield enhancing composition comprises a protease and at least one, at least two, at least three, at least four, or at least five different fungal strains.

The yield enhancing composition may include at least one, least two, at least three, at least four, or at least five different types of microorganisms. The microorganisms may be fungal strains, yeast strains, bacterial strains, and/or archaeal strains.

In an embodiment, the yield enhancing composition comprises at least one bacterial strain. In an embodiment, the yield enhancing composition comprises at least two bacterial strains. In an embodiment, the yield enhancing composition comprises at least three bacterial strains. In an embodiment, the yield enhancing composition comprises at least four bacterial strains. In an embodiment, the yield enhancing composition comprises at least five bacterial strains.

Examples of suitable bacterial strains include strains from the genera Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

In an embodiment, the at least one bacterial strain comprises a lactic acid bacterial strain. Suitable lactic acid bacterial strains include, but are not limited to, Lactococcus lactis ssp. lactis ATCC 19435, Lactococcus lactis ssp./lactis AS211, Lactobacillus delbrueckii ssp. delbrueckii ATCC 9649, Lactobacillus delbrueckii ssp. bulgaricus DSM 20081, Streptococcus salivarius subsp. thermophilus, Lactobacillus helveticus (thermophilic), Lactobacillus acidophilus, Lactobacillus bulgaricus, Pediococcus acidilactici, Streptococcus thermophilus, Streptococcus spp., Enterococcus spp, Pediococcus spp, Aerococcus spp, Leuconostoc spp, Lactobacillus salivarius, Lactobacillus brevis such as Lactobacillus brevis (DSM 23231), Lactobacillus fermentum, Lactobacillus reuteri, Lactobacillus hilgardii, Lactobacillus coryniformis, Lactobacillus curvatus, Lactobacillus plantarum such as Lactobacillus plantarum (DSM 19457), Lactobacillus casei, Lactobacillus curvatus, Leuconostoc mesenteroides sub-sp. cremoris, Lactococcus lactis, Enterococcus faecium, Lactobacillus kefir such as Lactobacillus kefir (DSM 19455), Lactobacillus buchneri, Lactobacillus paracasei, Lactobacillus diovilorans, Pediococcus pentosaceus, Lactobacillus rhamnosus, Pediococcus parvulus and Propionibacterium acidipropionici. In an embodiment the bacterial culture comprises the commercially available product called BioStabil® Biogas.

In an embodiment, the at least one bacterial strain is heterofermentative. The term “heterofermentative” as used herein means that the bacterial strain is capable of fermenting glucose into a variety of organic acids, like lactic acid, acetic acid, formic acid, citric acid, succinic acid and carbon dioxide. Heterofermentative bacterial strains include, but are not limited to, Leuconostoc spp, Lactobacillus salivarius, Lactobacillus brevis, Lactobacillus fermentum, Lactobacillus reuteri, Lactobacillus hilgardii, Lactobacillus coryniformis, Lactobacillus curvatus, Lactobacillus plantarum, Lactobacillus casei, Lactobacillus curvatus, Oenococcus, Weissella or any of the other bacterial cultures as described herein that are “heterofermentative”.

In a preferred embodiment, the yield enhancing composition comprises at least one heterofermentative bacterial strain. In a preferred embodiment, the yield enhancing composition comprises at least one heterofermentative bacterial strain and at least one non-heterofermentative bacterial strain.

In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five different types of bacterial strains selected from the genera Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

In an embodiment, the yield enhancing composition comprises at least one bacterial strain of the genus Bacillus. Examples of suitable Bacillus strains include Bacillus amyloliquefaciens, Bacillus megaterium, Bacillus pumilus, Bacillus subtilis, and/or Bacillus thuringiensis.

An exemplary Pseudomonas strain is Pseudomonas monteilii. In an embodiment, the bacterial strain is a Bacillus spp. strain. In an embodiment, the bacterial strain is a Pseudomonas spp. strain. In an embodiment, the bacterial strains comprise a Bacillus spp. strain and a Pseudomonas spp. strain. In an embodiment, the bacterial strain is Bacillus amyloliquefaciens. In an embodiment, the bacterial strain is Bacillus megaterium. In an embodiment, the bacterial strain is Bacillus subtilis. In an embodiment, the bacterial strain is Bacillus thuringiensis. In an embodiment, the bacterial strains comprise Bacillus amyloliquefaciens and Bacillus megaterium strains. In an embodiment, the bacterial strains comprise Bacillus amyloliquefaciens and Bacillus subtilis strains. In an embodiment, the bacterial strains comprise Bacillus amyloliquefaciens and Bacillus thuringiensis strains. In an embodiment, the bacterial strains comprises Bacillus amyloliquefaciens, Bacillus megaterium and Bacillus subtilis strains. In an embodiment, the bacterial strains comprise Bacillus amyloliquefaciens, Bacillus megaterium and Bacillus thuringiensis strains. In an embodiment, the bacterial strains comprise Bacillus amyloliquefaciens, Bacillus subtilis and Bacillus thuringiensis strains. In an embodiment, the bacterial strains comprise Bacillus amyloliquefaciens, Bacillus megaterium, Bacillus subtilis and Bacillus thuringiensis strains. In an embodiment, the bacterial strain is Pseudomonas monteilii. In an embodiment, the bacterial strains comprise Bacillus amyloliquefaciens, Bacillus megaterium, Bacillus subtilis, and Pseudomonas monteilii strains. In an embodiment, the bacterial strains comprise Bacillus amyloliquefaciens, Bacillus megaterium, Bacillus thuringiensis, and Pseudomonas monteilii strains. In an embodiment, the bacterial strains comprise Bacillus amyloliquefaciens, Bacillus subtilis, Bacillus thuringiensis, and Pseudomonas monteilii strains. In an embodiment, the bacterial strains comprise Bacillus amyloliquefaciens, Bacillus megaterium, Bacillus subtilis, Bacillus thuringiensis, and Pseudomonas monteilii strains.

In a preferred embodiment, the yield enhancing composition comprises a cellulase, amylase, protease and lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises a cellulase, amylase, protease and lipase, and the bacterial strains Bacillus amyloliquefaciens, Bacillus megaterium, Bacillus pumilus, Bacillus subtilis, and Bacillus thuringiensis.

In a preferred embodiment, the yield enhancing composition comprises a cellulase, amylase, protease and lipase, and at least one bacterial strain of the genus Pseudomonas.

In a preferred embodiment, the yield enhancing composition comprises a cellulase, amylase, protease and lipase, and Pseudomonas monteilii.

In a preferred embodiment, the yield enhancing composition comprises a cellulase, amylase, protease and lipase, and bacterial strains of the genera Bacillus and Pseudomonas.

In a preferred embodiment, the yield enhancing composition comprises a cellulase, amylase, protease and lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, alpha-amylase, protease a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises a cellulase, alpha-amylase, protease a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises a cellulase, alpha-amylase, protease a triacylglycerol lipase, and at least one bacterial strain of the genus Pseudomonas.

In a preferred embodiment, the yield enhancing composition comprises a cellulase, alpha-amylase, protease a triacylglycerol lipase, and Pseudomonas monteilii.

In a preferred embodiment, the yield enhancing composition comprises a cellulase, alpha-amylase, protease a triacylglycerol lipase, and bacterial strains of the genera Bacillus and Pseudomonas.

In a preferred embodiment, the yield enhancing composition comprises a cellulase, alpha-amylase, protease a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and at least one bacterial strain of the genus Pseudomonas.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Pseudomonas monteilii.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and bacterial strains of the genera Bacillus and Pseudomonas.

In a preferred embodiment, the yield enhancing composition comprises at least one, at least two, at least three cellulases selected from the group consisting of beta-glucosidase, cellobiohydrolase, and endoglucanase, an alpha-amylase, a protease and a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one bacterial strain selected from the group consisting of Bacillus amyloliquefaciens, Bacillus megaterium, Bacillus pumilus, Bacillus subtilis, Bacillus thuringiensis, and any combination thereof.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Bacillus amyloliquefaciens, Bacillus megaterium, Bacillus pumilus, Bacillus subtilis, and Bacillus thuringiensis.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and at least one bacterial strain of the genus Pseudomonas.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Pseudomonas monteilii.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and bacterial strains of the genera Bacillus and Pseudomonas.

In a preferred embodiment, the yield enhancing composition comprises at least one, at least two, at least three, at least four, or at least five cellulases selected from the group consisting of beta-glucosidase, cellobiohydrolase I, cellobiohydrolase II, endoglucanase I, and endoglucanase II, an alpha-amylase, a protease and a triacylglycerol lipase, and Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a hemicellulase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, a protease, a lipase, Bacillus amyloliquefaciens, Bacillus megaterium, Bacillus pumilus, Bacillus subtilis, Bacillus thuringiensis, and Pseudomonas monteilii.

In a preferred embodiment, the yield enhancing composition comprises a cellulase, a xylanase, a protease, a triacylglycerol lipase, Bacillus amyloliquefaciens, Bacillus megaterium, Bacillus pumilus, Bacillus subtilis, Bacillus thuringiensis, and Pseudomonas monteilii.

In an embodiment, the yield enhancing composition comprises at least one fungal strain. Examples of suitable fungal strains include strains of the genera Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton.

In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five fungal strains of the genera Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton.

Examples of suitable Aspergillus strains include, but are not limited to, Aspergillus niger, Aspergillus flavus, Aspergillus ustus, and Aspergillus wentii.

Examples of suitable Rhizopus fungal strains include, but are not limited to, Rhizopus arrhizus and Rhizopus oryzae.

Examples of suitable Trichodmera fungal strains include, but are not limited to, Trichoderma inhamatum, Trichoderma reesei, and Talaromyces emersonii.

In an embodiment, the yeast enhancing composition comprises at least one yeast strain. Examples of suitable yeast strains include, but are not limited to, Candida lignohabitans, Candida catenula, Candida guilliermondii, Yarrowia lipolytica, Candida tropicalis, Kluyveromyces, and Saccharomyces.

In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five yeast strains selected from Candida lignohabitans, Candida catenula, Candida guilliermondii, Yarrowia lipolytica, Candida tropicalis, Kluyveromyces, and Saccharomyces.

An exemplary Mucor strain is Mucor racemosus. An exemplary Paecilomyces strain is Paecilomyces lilacinus. An exemplary Trichoderma strain is Trichoderma inhamatum.

In an aspect, the fungal strain comprises an Aspergillus spp. strain. In an embodiment, the fungal strain comprises a Mucor spp. strain. In an embodiment, the fungal strain comprises a Paecilomyces spp. strain. In an embodiment, the fungal strain comprises a Trichoderma spp. strain. In an embodiment, the fungal strains comprise Aspergillus spp. and Mucor spp. strains. In an embodiment, the fungal strains comprise Aspergillus spp. and Paecilomyces spp. strains. In an embodiment, the fungal strains comprise Aspergillus spp. and Trichoderma spp. strains. In an embodiment, the fungal strain comprise Mucor spp. and Paecilomyces spp. strains. In an embodiment, the fungal strains comprise Mucor spp. and Trichoderma spp. strains. In an embodiment, the fungal strains comprise Paecilomyces spp. and Trichoderma spp. strains. In an embodiment, the fungal strain comprise Paecilomyces spp. and Trichoderma spp. strains. In an embodiment, the fungal strains comprise Mucor spp., Paecilomyces spp., and Trichoderma spp. strains. In an embodiment, the fungal strains comprise Aspergillus spp., Mucor spp., and Paecilomyces spp. strains. In an embodiment, the fungal strains comprise Aspergillus spp., Mucor spp., and Trichoderma spp. strains. In an embodiment, the fungal strains comprise Aspergillus spp., Mucor spp., Paecilomyces spp., and Trichoderma spp. strains. In an embodiment, the fungal strain is Aspergillus ustus. In an embodiment, the fungal strain is Mucor racemosus. In an embodiment, the fungal strain is Paecilomyces lilacinus. In an embodiment, the fungal strain is Trichoderma inhamatum. In an embodiment, the fungal strains comprise Mucor racemosus and Paecilomyces lilacinus strains. In an embodiment, the fungal strains comprise Mucor racemosus and Aspergillus ustus strains. In an embodiment, the fungal strains comprise Mucor racemosus and Trichoderma inhamatum strains. In an embodiment, the fungal strains comprise Paecilomyces lilacinus and Aspergillus ustus strains. In an embodiment, the fungal strains comprise Paecilomyces lilacinus and Trichoderma inhamatum strains. In an embodiment, the fungal strains comprise Aspergillus ustus and Trichoderma inhamatum strains. In an embodiment, the fungal strains comprise Mucor racemosus, Paecilomyces lilacinus, and Aspergillus ustus strains. In an embodiment, the fungal strains comprise Mucor racemosus, Paecilomyces lilacinus, and Trichoderma inhamatum strains. In an embodiment, the fungal strains comprise Paecilomyces lilacinus, Aspergillus ustus and Trichoderma inhamatum strains. In an embodiment, the fungal strains comprise Mucor racemosus, Paecilomyces lilacinus, Aspergillus ustus and Trichoderma inhamatum strains.

In an embodiment, the yield enhancing composition comprises at least one fungal strain and at least one bacterial strain. In an embodiment, the yield enhancing composition comprises at least two fungal strains and at least one bacterial strain. In an embodiment, the yield enhancing composition comprises at least three fungal strains and at least one bacterial strain. In an embodiment, the yield enhancing composition comprises at least four fungal strains and at least one bacterial strain. In an embodiment, the yield enhancing composition comprises at least one fungal strain and at least two bacterial strains. In an embodiment, the yield enhancing composition comprises at least one fungal strain and at least three bacterial strains. In an embodiment, the yield enhancing composition comprises at least one fungal strain and at least four bacterial strains. In an embodiment, the yield enhancing composition comprises at least two fungal strains and at least two bacterial strains. In an embodiment, the yield enhancing composition comprises at least three fungal strains and at least two bacterial strains. In an embodiment, the yield enhancing composition comprises at least four fungal strains and at least two bacterial strains. In an embodiment, the yield enhancing composition comprises at least five fungal strains and at least two bacterial strains. In an embodiment, the yield enhancing composition comprises at least three fungal strains and at least two bacterial strains. In an embodiment, the yield enhancing composition comprises at least two fungal strains and at least three bacterial strains. In an embodiment, the yield enhancing composition comprises at least two fungal strains and at least four bacterial strains. In an embodiment, the yield enhancing composition comprises at least two fungal strains and at least five bacterial strains. In an embodiment, the yield enhancing composition comprises at least three fungal strains and at least three bacterial strains. In an embodiment, the yield enhancing composition comprises at least four fungal strains and at least three bacterial strains. In an embodiment, the yield enhancing composition comprises at least five fungal strains and at least three bacterial strains. In an embodiment, the yield enhancing composition comprises at least three fungal strains and at least four bacterial strains. In an embodiment, the yield enhancing composition comprises at least three fungal strains and at least five bacterial strains. In an embodiment, the yield enhancing composition comprises at least four fungal strains and at least four bacterial strains. In an embodiment, the yield enhancing composition comprises at least five fungal strains and at least four bacterial strains. In an embodiment, the yield enhancing composition comprises at least four fungal strains and at least five bacterial strains. In an embodiment, the yield enhancing composition comprises at least five fungal strains and at five three bacterial strains.

In an embodiment, the yield enhancing composition comprises at least one fungal strain, at least one yeast strain, and at least one bacterial strain. In an embodiment, the yield enhancing composition comprises at least two, at least three, at least four, at least five, at least six, at least seven, at least eight, at least nine, or at least 10 different types of enzymes selected from the group consisting an amylase, cellulases, such as a cellobiohydrolase, an endo-p-1,4-glucanase, and/or a beta-glucosidase, beta-glucanases, such as a beta-(1,3)-glucanase, a beta-(1,4)-glucanase a beta-(1,3)(1,4)-glucanase, and/or a beta-(1,6)-glucanase, hemicellulases, for example, an endoxylanase, a beta-xylosidase, a arabinofuranosidase (e.g., alpha-L-arabionofuranosidase), a glucuronidase (e.g., an alpha-D-glucuronidase), an acetyl xylan esterase, a feruloyl esterase, a coumaroyl esterase, an alpha-galactosidase, a beta-galactosidase, a beta-mannanase and/or a beta-mannosidase, a lytic polysaccharide monooxygenase (e.g. GH61), pectinases, for example, an endo-polygalacturonase, a pectin-methyl esterase, an endo-galactanase, a beta-galactosidase, a pectin-acetyl esterase, an endo-pectin lyase, pectate lyase, alpha-rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase, a protease, a lipase, a phytase, a ligninase, a hexosyltransferase, a glucuronidase, an expansin, a cellulose induced protein or a cellulose integrating protein or like protein, a lactase, a sucrase, a catalase, and at least one, at least two, at least three, at least four, or at least five different microorganisms selected from the group consisting of yeast strains, fungal strains, and bacterial strains, wherein the yeast strains are selected from Candida lignohabitans, Candida catenula, Candida guilliermondii, Yarrowia lipolytica, Candida tropicalis, Kluyveromyces, and Saccharomyces, wherein the fungal strains are selected from Acremonium, Agahcus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton, and wherein the bacterial strains are selected from Bacillus, Pseudomonas, Acetobacter, Gluconobacter, Lactococcus, Lactobacillus, Pediococcus, Streptococcus, Aerococcus spp, Leuconostoc spp, Enterococcus and Propionibacterium.

The invention is further summarized in the following paragraphs:

1. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) producing biogas using the biogas unit,
wherein a yield enhancing composition comprising at least one, at least two, at least three, at least four, or at least five different types of enzymes and/or at least one, at least two, at least three, at least four, or at least five different microorganism strains is added to the biogas unit.
   2. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to the biogas unit; and
g) producing biogas using the biogas unit,
wherein a yield enhancing composition comprising at least one, at least two, at least three, at least four, or at least five different types of enzymes and/or at least one, at least two, at least three, at least four, or at least five different microorganism strains is added to the biogas unit.
   3. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to the biogas unit; and
g) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the whole stillage that is fed to the biogas unit.
   4. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a),
g) producing biogas using the biogas unit,
wherein a yield enhancing composition comprising at least one, at least two, at least three, at least four, or at least five different types of enzymes and/or at least one, at least two, at least three, at least four, or at least five different microorganism strains is added to the whole stillage that is fed to slurrying step a).
   5. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and/or to the biogas unit;
g) producing biogas using the biogas unit,
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit.
   6. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the slurrying step a); and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage that is fed to slurrying step a).
   7. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the biogas unit a); and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage that is fed to to the biogas unit.
   8. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage that is fed to slurrying step a) and/or to the biogas unit.
   9. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a), to the biogas unit, and to a solid-liquid separation step to generate thin stillage and wet cake;
g) feeding the the thin stillage to the slurrying step in a); and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit; and/or to the thin stillage that is fed to the slurrying step a).
   10. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a), to the biogas unit, and to a solid-liquid separation step to generate thin stillage and wet cake;
g) feeding the the thin stillage to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit; and/or to the thin stillage that is fed to the biogas unit.
   11. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a), to the biogas unit, and to a solid-liquid separation step to generate thin stillage and wet cake;
g) feeding the the thin stillage to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit; and/or to the thin stillage that is fed to the slurrying step a) and/or the thin stillage that is fed to the biogas unit.
   12. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake; and
g) producing biogas using the biogas unit,
wherein the thin stillage is used to make oil, such as corn oil, and residual materials resulting from purification of the oil;
wherein the residual materials resulting from purification of the corn oil are fed to the biogas unit; and
wherein a yield enhancing composition comprising at least one, at least two, at least three, at least four, or at least five different types of enzymes and/or at least one, at least two, at least three, at least four, or at least five different microorganism strains is added to the residual materials that are fed to the biogas unit.
   13. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage from step f) to slurrying step a) and/or to the biogas unit;
h) producing biogas using the biogas unit,
wherein the thin stillage is used to make oil, such as corn oil, and residual materials resulting from purification of the oil;
wherein the residual materials resulting from purification of the corn oil are fed to the biogas unit; and
wherein a yield enhancing composition comprising at least one, at least two, at least three, at least four, or at least five different types of enzymes and/or at least one, at least two, at least three, at least four, or at least five different microorganism strains is added to the the thin stillage fed to the slurrying step a), to the thin stillage fed to the biogas unit, and/or to the residual materials that are fed to the biogas unit.
   14. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to slurrying step a) and/or to the biogas unit and/or to a solid-liquid separation step to generate thin stillage and wet cake;
g) feeding the thin stillage from step f) to slurrying step a) and/or to the biogas unit;
h) producing biogas using the biogas unit,
wherein the thin stillage is used to make oil, such as corn oil, and residual materials resulting from purification of the oil;
wherein the residual materials resulting from purification of the corn oil are fed to the biogas unit; and
wherein a yield enhancing composition comprising at least one, at least two, at least three, at least four, or at least five different types of enzymes and/or at least one, at least two, at least three, at least four, or at least five different microorganism strains is added to the whole stillage that is fed to the slurrying step a), and/or to the whole stillage that is fed to the biogas unit and/or to the thin stillage fed to the slurrying step a) and/or to the thin stillage fed to the biogas unit, and/or to the residual materials that are fed to the biogas unit.
   15. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake; and
g) producing biogas using the biogas unit,
wherein the thin stillage is used to produce a protein product with a raw protein content of more than 44 percent and residual materials resulting from purification of the protein product;
wherein the residual materials resulting from purification of the protein product are fed to the biogas unit; and
wherein a yield enhancing composition comprising at least one, at least two, at least three, at least four, or at least five different types of enzymes and/or at least one, at least two, at least three, at least four, or at least five different microorganism strains is added to the residual materials resulting from purification of the protein product that are fed to the biogas unit.
   16. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage from step f) to slurrying step a) and/or to the biogas unit;
h) producing biogas using the biogas unit,
wherein the thin stillage is used to produce a protein product with a raw protein content of more than 44 percent and residual materials resulting from purification of the protein product;
wherein the residual materials resulting from purification of the protein product are fed to the biogas unit; and
wherein a yield enhancing composition comprising at least one, at least two, at least three, at least four, or at least five different types of enzymes and/or at least one, at least two, at least three, at least four, or at least five different microorganism strains is added to the the thin stillage fed to the slurrying step a), to the thin stillage fed to the biogas unit, and/or to the residual materials that are fed to the biogas unit.
   17. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to slurrying step a) and/or to the biogas unit and/or to a solid-liquid separation step to generate thin stillage and wet cake;
g) feeding the thin stillage from step f) to slurrying step a) and/or to the biogas unit;
h) producing biogas using the biogas unit,
wherein the thin stillage is used to produce a protein product with a raw protein content of more than 44 percent and residual materials resulting from purification of the protein product;
wherein the residual materials resulting from purification of the protein product are fed to the biogas unit; and
wherein a yield enhancing composition comprising at least one, at least two, at least three, at least four, or at least five different types of enzymes and/or at least one, at least two, at least three, at least four, or at least five different microorganism strains is added to the whole stillage that is fed to the slurrying step a), and/or to the whole stillage that is fed to the biogas unit and/or to the thin stillage fed to the slurrying step a) and/or to the thin stillage fed to the biogas unit, and/or to the residual materials that are fed to the biogas unit.
   18. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the wet cake to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the wet cake that is fed to the biogas unit.
   19. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the wet cake to the slurrying step a); and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the wet cake that is fed to the slurrying step a).
   20. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the wet cake to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the wet cake that is fed to the slurrying step a) and/or to the wet cake fed to the biogas unit.
   21. A process for producing a fermentation product and biogas from a starch-containing material, comprising the steps of:
a) forming a slurry comprising milled starch-containing material and water optionally with whole stillage and/or thin stillage, and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and/or to the biogas unit, and/or to a solid-liquid separation step to generate thin stillage and wet cake;
g) feeding the the thin stillage to the slurrying step in a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit; and/or to the thin stillage that is fed to the slurrying step a) and/or to the thin stillage that is fed to the biogas unit; and/or
wherein a biomass other than the starch-containing material is optionally pretreated and incubated with a yield enhancing composition comprising at least two, at least three, at least four, or at least five different enzymes and/or at least one, at least two, at least three, at least four, or at least five different microorganism strains before being fed to liquefying step b), and/or saccharifying step c), and/or fermenting step d), and/or to the biogas unit.
   22. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to the biogas unit; and
g) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit.
   23. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to slurrying step a); and
g) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the slurrying step a).
   24. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to slurrying step a) and/or to the biogas unit; and
g) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the slurrying step a) and/or to the biogas unit.
   25. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage that is fed to the biogas unit.
   26. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the slurrying step a); and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage from f) that is fed to the biogas unit.
   27. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to a solid-liquid separation step to generate thin stillage and wet cake;
g) feeding the thin stillage to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the thin stillage from f) that is fed to the slurrying step a) and/or to the biogas unit.
   28. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and/or to the biogas unit and/or to a solid-liquid separation step to generate thin stillage and wet cake;
h) feeding the thin stillage to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the whole stillage from step e) that is fed to slurrying step a) and/or to the whole stillage that is fed to the biogas unit and/or to the thin stillage from f) that is fed to the slurrying step a) and/or to the thin stillage that is fed to the biogas unit.
   29. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and to the biogas unit,
wherein a yield enhancing composition comprising at least one, at least two, at least three, at least four, or at least five different types of enzymes and/or at least one, at least two, at least three, at least four, or at least five different microorganism strains is added to the outflow of the biogas unit before being fed to slurrying step a).
   30. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and to the biogas unit; wherein whole stillage from step e) is fed to a solid-liquid separation step to generate thin stillage and wet cake;
wherein the thin stillage is used to make oil, such as corn oil, and residual materials resulting from purification of the oil; wherein the residual materials resulting from purification of the corn oil are fed to the biogas unit;
wherein the residual materials resulting from purification of the corn oil are fed to the biogas unit; and
wherein the yield enhancing composition comprising at least two, at least three, at least four, or at least five different types of enzymes and/or at least one, at least two, at least three, at least four, or at least five different microorganism strains are added to the residual materials resulting from purification of the corn oil that are fed to the biogas unit.
   31. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and to the biogas unit;
g) producing biogas using the biogas unit;
wherein whole stillage from step e) is fed to a solid-liquid separation step to generate thin stillage and wet cake;
wherein the thin stillage is used to produce a protein product with a raw protein content of more than 44 percent and residual materials resulting from purification of the protein product.
wherein the residual materials resulting from purification of the protein product are fed to the biogas unit; and
wherein the yield enhancing composition comprising at least two, at least three, at least four, or at least five different types of enzymes and/or at least one, at least two, at least three, at least four, or at least five different microorganism strains are added to the residual materials resulting from purification of the protein product that are fed to the biogas unit.
   32. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to a solid-liquid separation step to generate thin stillage and wet cake;
g) feeding the wet cake to the biogas unit; and
h) producing biogas using the biogas unit;
wherein the yield enhancing composition comprising at least two, at least three, at least four, or at least five different types of enzymes and/or at least one, at least two, at least three, at least four, or at least five different microorganism strains is added to the wet cake that is fed to the biogas unit.
   33. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to a solid-liquid separation step to generate thin stillage and wet cake;
g) feeding the wet cake to the slurrying step a); and
h) producing biogas using the biogas unit;
wherein the yield enhancing composition comprising at least two, at least three, at least four, or at least five different types of enzymes and/or at least one, at least two, at least three, at least four, or at least five different microorganism strains is added to the wet cake that is fed to the slurrying step a).
   34. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to a solid-liquid separation step to generate thin stillage and wet cake;
g) feeding the wet cake to the slurrying step a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the wet cake that is fed to the slurrying step a) and/or to the wet cake fed to the biogas unit.
   35. A process for producing a fermentation product and biogas from a starch-containing material, comprising the steps of:
a) forming a slurry comprising milled starch-containing material and water with at least 0.1 t of dry matter in the form of whole stillage and/or thin stillage, and at least 0.1 m³ of outflow from a biogas unit per tonne of milled starch-containing material;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage; and
f) feeding the whole stillage from step e) to slurrying step a) and/or to the biogas unit, and/or to a solid-liquid separation step to generate thin stillage and wet cake;
g) feeding the the thin stillage to the slurrying step in a) and/or to the biogas unit; and
h) producing biogas using the biogas unit;
wherein a yield enhancing composition comprising at least one, at least two, at least three, at least four, or at least five different types of enzymes and/or at least one, at least two, at least three, at least four, or at least five different microorganism strains is added to the biogas unit and/or to the whole stillage that is fed to slurrying step a) and/or the whole stillage that is fed to the biogas unit; and/or to the thin stillage that is fed to the slurrying step a) and/or to the thin stillage that is fed to the biogas unit; and/or
wherein a biomass other than the starch-containing material is optionally pretreated and incubated with a yield enhancing composition comprising at least two, at least three, at least four, or at least five different enzymes and/or at least one, at least two, at least three, at least four, or at least five different microorganism strains before being fed to liquefying step b), and/or saccharifying step c), and/or fermenting step d), and/or to the biogas unit.
   36. The process of any one of paragraphs 1-35, wherein a biomass other than the starch-containing material is optionally pretreated and incubated with a yield enhancing composition comprising at least two, at least three, at least four, or at least five different enzymes and/or at least one, at least two, at least three, at least four, or at least five different microorganism strains before being fed to liquefying step b), saccharifying step c), and/or fermenting step d) and/or to the biogas unit.
   37. The process of any one of paragraphs 1-36, wherein a biomass other than the starch-containing material is fed to the biogas unit.
   38. The process of paragraphs 36 or 37, wherein the biomass comprises cellulosic material and/or lignocellulosic material and/or hemicellulosic material and/or starch material and/or pectin material and/or lipid material.
   39. The process of any one of paragraphs 35-38, wherein the biomass comprises pretreated corn stover.
   40. The process of any one of paragraphs 1-39, wherein the fermentation product is ethanol.
   41. The process of any one of paragraphs 1-40, wherein the starch-containing material comprises corn.
   42. The process of any one of paragraphs 1-41, wherein saccharifying step ii) and fermenting step iii) are performed simultaneously in a simultaneous saccharification and fermentation.
   43. The process of any one of paragraphs 1-42, wherein at least 0.2 t TS per tonne of milled starch-containing is added to the slurrying step in a) in the form of a mixture of thin stillage and whole stillage.
   44. The process of any one of paragraphs 1-43, wherein at least 0.12 t TS per tonne of milled starch-containing material is recycled to the slurrying step in a) in the form of whole stillage.
   45. The process of any one of paragraphs 1-44, wherein at least 0.1 t TS of thin stillage per t of mash is recycled to the slurrying step in a).
   46. The process of any one of paragraphs 1-45, wherein the milled starch-containing material in a) from a dry milling step has a mass fraction of at least 60 percent of particles with a particle size of <0.5 mm.
   47. The process of any one of paragraphs 1-46, wherein the ammonium nitrogen content in the biogas fermenters of the biogas unit is kept at between 6000-9000 ppm.
   48. The process of any one of paragraphs 1-47, wherein the mean hydraulic residence time for the biogas unit is at least 30 days.
   49. The process of any one of paragraphs 1-48, wherein the proportion of outflow from the biogas unit per tonne of milled starch-containing material in a) is at least 0.2 m³.
   50. The process of any one of paragraphs 1-49, wherein the distillation step in e), at least 400 liters of fermentation product, such as ethanol, are separated per tonne of milled grain.
   51. The process of any one of paragraphs 1-50, wherein the slurrying step in a), at least 100 g of ammonium nitrogen per tonne of milled grain is recycled via the outflow from the biogas unit.
   52. The process of any one of paragraphs 1-51, wherein the slurrying step in a), a maximum of 1000 g of ammonium nitrogen per tonne of milled starch-containing material is recycled via the outflow from the biogas unit.
   53. The process of any one of paragraphs 1-52, wherein less than 1.2 m³ of fresh water is used per tonne of milled starch-containing material.
   54. The process of any one of paragraphs 1-53, wherein at least 15 kg of glycerin per tonne of milled grain is recycled to the slurrying step via the whole stillage and thin stillage.
   55. The process of any one of paragraphs 1-54, wherein the at least two, at least three, at least four, or at least five different types of enzymes are selected from the group consisting of an amylase, a beta-glucanases, a cellulase, a hemicellulase, a lipase, a pectinase, and a protease.
   56. The process of any one of paragraphs 1-55, wherein the yield enhancing composition comprises a cellulase and a hemicellulase.
   57. The process of any one of paragraphs 1-56, wherein the yield enhancing composition comprises a cellulase and a xylanase.
   58. The process of any one of paragraphs 1-57, wherein the yield enhancing composition comprises a beta-glucanase and a pectinase.
   59. The process of any one of paragraphs 1-58, wherein the yield enhancing composition comprises cellulase, xylanase, protease and lipase.
   60. The process of any one of paragraphs 1-59, wherein the yield enhancing composition comprises at least one fungal strain.
   61. The process of any one of paragraphs 1-60, wherein the yield enhancing composition comprises at least one bacterial strain.
   62. The process of any one of paragraphs 1-61, wherein the yield enhancing composition comprises at least one fungal strain and at least one bacterial strain.
   63. The process of any one of paragraphs 1-62, wherein the yield enhancing composition comprises at least one bacterial strain of the genus Bacillus.
   64. The process of any one of paragraphs 1-63, wherein the yield enhancing composition comprises at least one bacterial strain selected from the group consisting of Bacillus amyloliquefaciens, Bacillus megaterium, Bacillus pumilus, Bacillus subtilis, Bacillus thuringiensis, and any combination thereof.
   65. The process of any one of paragraphs 1-64, wherein the yield enhancing composition comprises Bacillus amyloliquefaciens, Bacillus megaterium, Bacillus pumilus, Bacillus subtilis, and Bacillus thuringiensis.
   66. The process of any one of paragraphs 1-65, wherein the yield enhancing composition comprises at least one bacterial strain of the genus Pseudomonas.
   67. The process of any one of paragraphs 1-66, wherein the yield enhancing composition comprises Pseudomonas monteilii.
   68. The process of any one of paragraphs 1-67, wherein the yield enhancing composition comprises bacterial strains of the genera Bacillus and Pseudomonas.
   69. The process of any one of paragraphs 1-68, wherein the yield enhancing composition comprises Bacillus amyloliquefaciens, Bacillus megaterium, Bacillus pumilus, Bacillus subtilis, Bacillus thuringiensis, and Pseudomonas monteilii.
   70. The process of any one of paragraphs 1-69, wherein the yield enhancing composition comprises a cellulase, a hemicellulase, Bacillus amyloliquefaciens, Bacillus megaterium, Bacillus pumilus, Bacillus subtilis, Bacillus thuringiensis, and Pseudomonas monteilii.
   71. The process of any one of paragraphs 1-70, wherein the yield enhancing composition comprises a cellulase, a xylanase, Bacillus amyloliquefaciens, Bacillus megaterium, Bacillus pumilus, Bacillus subtilis, Bacillus thuringiensis, and Pseudomonas monteilii.
   72. The process of any one of paragraphs 1-71, wherein the yield enhancing composition comprises a cellulase, a xylanase, a protease, a lipase, Bacillus amyloliquefaciens, Bacillus megaterium, Bacillus pumilus, Bacillus subtilis, Bacillus thuringiensis, and Pseudomonas monteilii.
   73. The process of any one of paragraphs 1-72, wherein the yield enhancing composition comprises a cellulase, a xylanase, a protease, a triacylglycerol lipase, Bacillus amyloliquefaciens, Bacillus megaterium, Bacillus pumilus, Bacillus subtilis, Bacillus thuringiensis, and Pseudomonas monteilii.
   74. The process of any one of paragraphs 1-73, wherein the thin stillage is mixed with pig manure and the yield enhancing composition is added to the mixture and the mixture is fed to the biogas unit.
   75. The process of any one of paragraphs 1-73, wherein the thin stillage is mixed with corn sillage and the yield enhancing composition is added to the mixture and the mixture is fed to the biogas unit.
   76. The process of any one of paragraphs 1-73, wherein the thin stillage is mixed with food waste and the yield enhancing composition is added to the mixture and the mixture is fed to the biogas unit.
   77. The process of any one of paragraphs 1-73, wherein the thin stillage is mixed with municipal solid waste and the yield enhancing composition is added to the mixture and the mixture is fed to the biogas unit.
   78. The process of any one of paragraphs 1-73, wherein the thin stillage is mixed with cassava pulp and the yield enhancing composition is added to the mixture and the mixture is fed to the biogas unit.
   79. The process of any one of paragraphs 1-73, wherein the thin stillage is mixed with sugar beet pulp and the yield enhancing composition is added to the mixture and the mixture is fed to the biogas unit.
   80. The process of any one of paragraphs 1-73, wherein the thin stillage is mixed with grass clippings and the yield enhancing composition is added to the mixture and the mixture is fed to the biogas unit.
   81. The process of any one of paragraphs 1-73, wherein the thin stillage is mixed with straw pellets and the yield enhancing composition is added to the mixture and the mixture is fed to the biogas unit.
   82. The process of any one of paragraphs 1-73, wherein the thin stillage is mixed with pretreated corn stover and the yield enhancing composition is added to the mixture and the mixture is fed to the biogas unit.
   83. The process of any one of paragraphs 1-82, wherein the yield enhancing composition comprises a cellulase.
   84. The process of any one of paragraphs 1-83, wherein the yield enhancing composition comprises a beta-glucosidase.
   85. The process of any one of paragraphs 1-84, wherein the yield enhancing composition comprises an endoglucanase.
   86. The process of any one of paragraphs 1-85, wherein the yield enhancing composition comprises a cellobiohydrolase.
   87. The process of any one of paragraphs 1-86, wherein the yield enhancing composition comprises an endoglucanase, a beta-glucosidase and a cellobiohydrolase.
   88. The process of any one of paragraphs 1-87, wherein the yield enhancing composition comprises a beta-glucosidase, a cellobiohydrolase I, a cellobiohydrolase II, an Auxillary Activity Family 9 (AA9) polypeptide, a xylanase and a beta-xylosidase.
   89. The process of any one of paragraphs 1-88, wherein the yield enhancing composition comprises a beta-glucosidase, a cellobiohydrolase I, a cellobiohydrolase II, an AA9 polypeptide, a xylanase, a beta-xylosidase, and a protease.
   90. The process of any one of paragraphs 1-89, wherein the yield enhancing composition comprises a beta-glucosidase, a cellobiohydrolase, an endoglucanase, an arabinofuranosidase, and a xylanase.
   91. The process of any one of paragraphs 1-90, wherein the yield enhancing composition comprises a beta-glucosidase, a cellobiohydrolase, an endoglucanase, an arabinofuranosidase, a xylanase and a protease.
   92. The process of any one of paragraphs 1-91, wherein the yield enhancing composition comprises a lipase.
   93. The process of any one of paragraphs 1-92, wherein the yield enhancing composition comprises a protease.
   94. The process of any one of paragraphs 1-93, wherein the yield enhancing composition comprises a cellulase and a xylanase.
   95. The process of any one of paragraphs 1-94, wherein the yield enhancing composition comprises a beta-glucosidase and a xylanase.
   96. The process of any one of paragraphs 1-95, wherein the yield enhancing composition comprises an endoglucanase and a xylanase.
   97. The process of any one of paragraphs 1-96, wherein the yield enhancing composition comprises a cellobiohydrolase and a xylanase.
   98. The process of any one of paragraphs 1-97, wherein the yield enhancing composition comprises an endoglucanase, a beta-glucosidase, a cellobiohydrolase and a xylanase.
   99. The process of any one of paragraphs 1-98, wherein the yield enhancing composition comprises an amylase.
   100. The process of any one of paragraphs 1-99, wherein the yield enhancing composition comprises an amylase and a glucoamylase.
   101. The process of any one of paragraphs 1-100, wherein the yield enhancing composition comprises a beta-glucanase.
   102. The process of any one of paragraphs 1-101, wherein the yield enhancing composition comprises a pectinase.
   103. The process of any one of paragraphs 1-102, wherein the yield enhancing composition comprises a beta-glucanase and a pectinase.
   104. The process of any one of paragraphs 1-103, wherein the yield enhancing composition comprises a cellulase, a xylanase, a protease, and a triacylglycerol lipase.
   105. The process of any one of paragraphs 1-104, wherein the yield enhancing composition comprises bacterial strains of the genera Bacillus and Pseudomonas.
   106. The process of any one of paragraphs 1-105, wherein the yield enhancing composition comprises Bacillus amyloliquefaciens, Bacillus megaterium, Bacillus pumilus, Bacillus subtilis, Bacillus thuringiensis, and Pseudomonas monteilii.
   107. The process of any one of paragraphs 1-106, wherein the yield enhancing composition comprises a cellulase, a hemicellulase, Bacillus amyloliquefaciens, Bacillus megaterium, Bacillus pumilus, Bacillus subtilis, Bacillus thuringiensis, and Pseudomonas monteilii.
   108. The process of any one of paragraphs 1-107, wherein the yield enhancing composition comprises a cellulase, a xylanase, Bacillus amyloliquefaciens, Bacillus megaterium, Bacillus pumilus, Bacillus subtilis, Bacillus thuringiensis, and Pseudomonas monteilii.
   109. The process of any one of paragraphs 1-108, wherein the yield enhancing composition comprises a cellulase, a xylanase, a protease, a lipase, Bacillus amyloliquefaciens, Bacillus megaterium, Bacillus pumilus, Bacillus subtilis, Bacillus thuringiensis, and Pseudomonas monteilii.
   110. The process of any one of paragraphs 1-109, wherein the yield enhancing composition comprises a cellulase, a xylanase, a protease, a triacylglycerol lipase, Bacillus amyloliquefaciens, Bacillus megaterium, Bacillus pumilus, Bacillus subtilis, Bacillus thuringiensis, and Pseudomonas monteilii.
   111. The process of any one of paragraphs 1-110, wherein the yield enhancing composition is formulated to enhance fermentation product yield.
   112. The process of any one of paragraphs 1-111, wherein the yield enhancing composition is formulated to enhance ethanol yield.
   113. The process of any one of paragraphs 1-112, wherein the yield enhancing composition is formulated to enhance fermentation product yield and biogas yield.
   114. The process of any one of paragraphs 1-113, wherein the yield enhancing composition is formulated to enhance ethanol yield and biogas yield.
   115. The process of any one of paragraphs 1-114, wherein the yield enhancing composition is formulated to increase the rate of biogas production.

The present invention is further described by the following examples that should not be construed as limiting the scope of the invention.

### Examples

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### Materials

Yield Enhancing Composition 1: composition comprising cellulase (e.g., endoglucanase), alpha-amylase, protease, triacylglycerol lipase enzymes and Bacillus amyloliquefaciens, Bacillus megaterium, Bacillus pumilus, Bacillus subtilis, Bacillus thuringiensis, and Pseudomonas monteilii bacterial strains.

Yield Enhancing Composition 2: composition comprising endoglucanase.

Yield Enhancing Composition 3: composition comprising cellobiohydrolase.

Yield Enhancing Composition 4: composition comprising beta-glucosidase.

Yield Enhancing Composition 5: composition comprising beta-glucosidase, cellobiohydrolase, and endoglucanase.

Yield Enhancing Composition 6: composition comprising beta-glucosidase, cellobiohydrolase I, cellobiohydrolase II, AA9 polypeptide, xylanase and beta-xylosidase.

Yield Enhancing Composition 7: composition comprising beta-glucosidase, cellobiohydrolase I, cellobiohydrolase II, AA9 polypeptide, xylanase, beta-xylosidase, and protease.

Yield Enhancing Composition 8: composition comprising beta-glucosidase, cellobiohydrolase, endoglucanase, arabinofuranosidase, xylanase.

Yield Enhancing Composition 9: composition comprising beta-glucosidase, cellobiohydrolase, endoglucanase, arabinofuranosidase, xylanase and protease.

Yield Enhancing Composition 10: composition comprising lipase.

Yield Enhancing Composition 11: composition comprising protease.

Yield Enhancing Composition 12: composition comprising cellulase and xylanase.

Yield Enhancing Composition 13: composition comprising alpha-amylase.

Yield Enhancing Composition 14: composition comprising alpha-amylase and glucoamylase.

Yield Enhancing Composition 15: composition comprising beta-glucanase.

Yield Enhancing Composition 16: composition comprising pectinase.

Yield Enhancing Composition 17: composition comprising beta-glucanase and pectinase.

### Example 1: Thin Stillage

Bio Methane Potential tests (BMPs) are conducted in 500 mL anaerobic reactors utilizing AMPTS II systems from BPC Instruments. The AMPTS II instrument is an analytical device that measures ultra-low bio-methane flows from the anaerobic batch digestion of biodegradable substrates.

For each substrate, 9 different treatments listed in Table 1 are evaluated. Each treatment is run in triplicate in 500 mL anaerobic reactors. Reactors are fed with 300 grams of substrate and seeded with 60 grams of active sludge from an industrial anerobic digester processing cow manure. Enzyme and microbial treatments are added at a rate of 0.1% weight % of treatment product vs. dry mass of substrate.

Gas production is monitored by the AMPTS for 45 days. Gas is recirculated through a handheld gas analyzer weekly to determine the concentration of CO2 in order to calculate the remaining concentration and volume of methane produced. Differences in methane production are evaluated on the basis of methane yield per gram of volatile substrate (mL CH4/g VS). Total solids (TS) is determined by drying at 105 degree Celsius until no further weight change occurs. Ash is determined in a furnace by heating the sample to 550 degree Celsius in a crucible until no further weight change occurs. Volatile solids (VS) is calculated by subtracting total solids with ash content.

The enzyme and microbial treatments are tested for their ability to provide an improved biogas yield and/or increased rate of biogas production. The list of enzymes and microbial products tested is shown in Table 1.

TABLE 1

| Treatment | Addition rate * |
|---|---|
| Control | N.A. |
| Yield Enhancing Composition 1 | 0.1-0.2% wt. product to wt. TS substrate |
| Yield Enhancing Composition 2 | 0.1-0.2% wt. product to wt. TS substrate |
| Yield Enhancing Composition 3 | 0.1-0.2% wt. product to wt. TS substrate |
| Yield Enhancing Composition 4 | 0.1-0.2% wt. product to wt. TS substrate |
| Yield Enhancing Composition 5 | 0.1-0.2% wt. product to wt. TS substrate |
| Yield Enhancing Composition 6 | 0.1-0.2% wt. product to wt. TS substrate |
| Yield Enhancing Composition 7 | 0.1-0.2% wt. product to wt. TS substrate |
| Yield Enhancing Composition 8 | 0.1-0.2% wt. product to wt. TS substrate |
| Yield Enhancing Composition 9 | 0.1-0.2% wt. product to wt. TS substrate |
| Yield Enhancing Composition 10 | 0.1-0.2% wt. product to wt. TS substrate |
| Yield Enhancing Composition 11 | 0.1-0.2% wt. product to wt. TS substrate |
| Yield Enhancing Composition 12 | 0.1-0.2% wt. product to wt. TS substrate |
| Yield Enhancing Composition 13 | 0.1-0.2% wt. product to wt. TS substrate |
| Yield Enhancing Composition 14 | 0.1-0.2% wt. product to wt. TS substrate |
| Yield Enhancing Composition 15 | 0.1-0.2% wt. product to wt. TS substrate |
| Yield Enhancing Composition 16 | 0.1-0.2% wt. product to wt. TS substrate |
| Yield Enhancing Composition 17 | 0.1-0.2% wt. product to wt. TS substrate |

* Addition rates are expressed in % wt of the product to wt. of the total solids (TS) of substrate Thin stillage from an industrial ethanol plant is obtained.
Total solids of the thin stillage is approximately 6% w/w solids.
60 grams of active sludge from an industrial anerobic digester processing cow manure is added to 300 grams of thin stillage.
Treatments are added as presented in Table 1.
Each treatment is conducted in triplicate in 500 mL anaerobic reactors.
Results to be evaluated: The extent to which the treatment increases methane yield is evaluated on the basis of methane volume per gram of volatile substrate (mL CH4/g VS) on a daily basis from the start of digestion to 45 days.
The extent to which the treatment increases the rate of biogas production is increased at 7, 14, 28 or 30 day time points is also evaluated.

### Example 2: Whole Stillage

Whole stillage from a commercial ethanol plant is obtained. Total solids of the whole stillage is approximately 10% w/w solids with approximately 65% volatile solids. 60 grams of active sludge from an industrial anerobic digester processing cow manure is added to 300 grams of whole stillage. Treatments are added as presented in Table 1. Each treatment is conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28 or 30 day time points is also evaluated.

### Example 3: Defatted Syrup

Defatted syrup from an industrial corn to ethanol plant is obtained. Total solids of the defatted syrup is approximately 35% w/w solids with approximately 60% volatile solids. The defatted syrup is diluted 1:1 with water to create a diluted defatted syrup. 60 grams of active sludge from an industrial anerobic digester processing cow manure is added to 300 grams of diluted defatted syrup. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 4: Thin Stillage Codigested with Pig Manure

Thin stillage from a commercial ethanol plant are obtained. Pig manure is obtained from an industrial pig farm. Thin stillage is combined with pig manure to form a thin stillage and pig manure mixture. Active sludge from an industrial anerobic digester processing cow manure will be added to the thin stillage and pig manure mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 5: Thin Stillage Codigested with Corn Sillage

Thin stillage from a commercial ethanol plant are obtained. Corn Sillage is obtained from an industrial corn farm. Thin stillage will be combined with Corn Sillage to form a thin stillage and corn sillage mixture. Active sludge from an industrial anerobic digester processing cow manure is added to the thin stillage and corn sillage mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 6: Thin Stillage with Codigested Mixed Food Waste

Thin stillage from a commercial ethanol plant will be obtained. Mixed Food Waste will be obtained from an industrial biogas plant. Thin stillage will be combined with Mixed Food Waste to form a thin stillage and Mixed Food Waste mixture. Active sludge from an industrial anerobic digester processing cow manure will be added to the thin stillage and mixed food waste mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 7: Thin Stillage with Codigested Municipal Solid Waste

Thin stillage from a commercial ethanol plant is obtained. Municipal Solid Waste is obtained from a commercial Biogas plant. Thin stillage is combined with Municipal Solid Waste to form a thin stillage and Municipal Solid Waste mixture. Active sludge from an industrial anerobic digester processing cow manure is added to the thin stillage and Municipal Solid Waste mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 8: Thin Stillage with Codigested Cassava Pulp

Thin stillage from an industrial corn to ethanol plant are obtained. Cassava Pulp is obtained from a Cassava Starch Manufacturing plant. Thin stillage is combined with cassava pulp to form a thin stillage and cassava pulp mixture. Active sludge from an industrial anerobic digester processing cow manure is added to the thin stillage and cassava pulp mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 9: Thin Stillage with Codigested Sugar Beet Pulp

Thin stillage from a commercial ethanol plant is obtained. Sugar beet pulp is obtained from a sugar beet refining plant. Thin stillage is combined with sugar beet pulp to form a thin stillage and sugar beet pulp mixture. Active sludge from an industrial anerobic digester processing cow manure is added to the thin stillage and sugar beet pulp mixture. Treatments are added as presented in Table 1. Experiments are conducted in triplicate in 500 mL anaerobic reactors. Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 10: Thin Stillage with Codigested Grass Clippings

Thin stillage from a commercial ethanol plant is obtained. Grass clipping are obtained from natural grass land. Thin stillage is combined with grass clippings to form a thin stillage and grass clippings mixture. Active sludge from an industrial anerobic digester processing cow manure is added to the thin stillage and grass clippings mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 11: Thin Stillage with Codigested Straw Pellets

Thin stillage from a commercial ethanol plant is obtained. Straw Pellets are obtained from a feed supply store. Thin stillage is combined with straw pellets to form a thin stillage and straw pellet mixture. Active sludge from an industrial anerobic digester processing cow manure will be added to the thin stillage and straw pellet mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

### Example 12: Thin Stillage Codigested with Pretreated Corn Stover

Thin stillage and pretreated corn stover are obtained from a commercial ethanol plant. Thin stillage will be combined with pretreated corn stover to form a thin stillage and pretreated corn stover mixture. Active sludge from an industrial anerobic digester processing cow manure is added to the thin stillage and corn sillage mixture. Treatments are added as presented in Table 1. Experiments are conducted in triplicate in 500 mL anaerobic reactors.

Results to be evaluated: The extent to which the treatment increases methane yield is evaluated on the basis of methane volume per gram of volatile substrate (mL CH4/g VS) on a daily basis from the start of digestion to 45 days. The extent to which the treatment increases the rate of biogas production is increased at 7, 14, 28, or 30 day time points is also evaluated.

1. A process for producing a fermentation product and biogas from a starch-containing material, comprising the steps of:
a) forming a slurry comprising milled starch-containing material and water with whole stillage and/or thin stillage and/or outflow from a biogas unit;
b) liquefying the starch-containing material from step i) at a temperature above the initial gelatinization temperature to produce dextrins;
c) saccharifying the dextrins to produce fermentable sugars;
d) fermenting the fermentable sugars to produce a beer comprising the fermentation product;
e) distilling the beer to produce the fermentation product and whole stillage;
f) feeding the whole stillage from step e) to slurrying step a) and/or to the biogas unit and/or to a solid-liquid separation step to generate thin stillage and wet cake; and/or
g) feeding the thin stillage to the slurrying step a) and/or to the biogas unit;
wherein a yield enhancing composition comprising at least one, at least two, at least three, at least four, or at least five different types of enzymes and/or at least one, at least two, at least three, at least four, or at least five different microorganism strains is:
i) added to the biogas unit;
ii) added to the whole stillage from e) that is fed to the slurrying step in a); and/or
iii) added to the whole stillage from e) that is fed to the biogas unit; and/or
iv) added to the outflow of the biogas unit before being fed to slurrying step a); and/or
iv) added to the thin stillage that is fed to the slurrying step; and/or
v) added to residual materials resulting from purification of the oil (e.g., corn) that are fed to the biogas unit; and/or
vi) added to the residual materials resulting from purification of the protein product that are fed to the biogas unit; and/or
vii) added to the wet cake that is fed to the slurrying step; and/or
viii) added to the wet cake that is fed to the biogas unit; and/or
ix) added to the biogas unit; and/or
x) biomass added to any one of i)-ix).

2. The process of claim 1, wherein the fermentation product is ethanol.

3. The process of claim 1, wherein the starch-containing material comprises corn.

4. The process of claim 1, wherein saccharifying step ii) and fermenting step iii) are performed simultaneously in a simultaneous saccharification and fermentation.

5. The process of claim 1, wherein whole stillage from step e) is fed to a solid-liquid separation step to generate thin stillage and wet cake.

6. The process of claim 5, wherein the thin stillage is fed to the slurrying step in a).

7. The process of claim 6, wherein the yield enhancing composition is added to the thin stillage that is fed to the mashing step in 1a).

8. The process of claim 5, wherein the thin stillage is used to make oil, such as corn oil, and residual materials resulting from purification of the oil.

9. The process of claim 8, wherein the residual materials resulting from purification of the corn oil are fed to the biogas unit.

10. The process of claim 9, wherein the yield enhancing composition is added to the residual materials resulting from purification of the corn oil that are fed to the biogas unit.

11. The process of claim 5, wherein the thin stillage is used to produce a protein product with a raw protein content of more than 44 percent and residual materials resulting from purification of the protein product.

12. The process of claim 11, wherein the residual materials resulting from purification of the protein product are fed to the biogas unit.

13. The process of claim 12, wherein the yield enhancing composition is added to the residual materials resulting from purification of the protein product that are fed to the biogas unit.

14. The process of claim 5, wherein the wet cake is fed to the slurrying in step a).

15. The process of claim 14, wherein the yield enhancing composition is added to the wet cake that is fed to the slurrying step in 1a).

16. The process of claim 5, wherein the wet cake is fed to the biogas unit.

17. The process of claim 16, wherein a yield enhancing composition is added to the wet cake that is fed to the biogas unit.

18. The process of claim 1, wherein a biomass other than the starch-containing material is optionally pretreated and incubated with the yield enhancing composition before being fed to liquefying step b), saccharifying step c), and/or fermenting step d)

19. The process of claim 1, wherein a biomass other than the starch-containing material is fed to the biogas unit.

20. The process of claim 1, wherein the biomass comprises pretreated corn stover.

21. (canceled)

22. (canceled)

23. (canceled)

24. (canceled)

25. (canceled)

26. (canceled)

27. (canceled)

28. (canceled)

29. (canceled)

30. The process of claim 1, wherein the yield enhancing composition is formulated to increase fermentation product yield (e.g., ethanol yield), biogas yield, the rate of biogas production, and combinations thereof.

31. The process of claim 1, wherein the thin stillage is mixed with an additional material obtained from a farm industrial plant or natural plant origin, to form a mixture, and wherein the yield enhancing composition is added to the mixture to form a combined mixture and the combined mixture is fed to the biogas unit.

32. The method of claim 31, wherein the additional material is pig manure, corn sillage, food waste, municipal solid waste, cassava pulp, sugar beet pulp, grass clippings, straw pellets, or pretreated corn stover.
